(19) United States
(12) Patent Application Publication
(10) Pub. No.: US 2026/0335514 A1
(43) Pub. Date: Oct. 29, 2026

(54) Drug-Coated Medical Devices & Methods of Making

(71) Applicant: Bard Peripheral Vascular, Inc., Franklin Lakes, NJ (US)

(72) Inventors: Vincent Sullivan, Cary, NC (US); Qihua Xu, Cary, NC (US); Charles D. Shermer, Raleigh, NC (US); Hiep Do, Chandler, AZ (US); Cyal Lecy, Buffalo, MN (US); Melissa Boyle, Phoenix, AZ (US); Edgar Sanchez Garcia, Tempe, AZ (US); Jeremy Cox, Salt Lake City, UT (US); Casey Rockwood, Phoenix, AZ (US); Song Ding, Apex, NC (US); Tuane Cristina Dos Santos, Cary, NC (US)

(21) Appl. No.: 19/101,579

(22) PCT Filed: Aug. 11, 2022
(86) PCT No.: PCT/US2022/074825

§ 371 Date: Feb. 5, 2025

(51) Int. Cl. A61L 31/10; A61L 31/16

(52) U.S. Cl. CPC A61L 31/10; A61L 31/16; A61L 2300/204; A61L 2300/21

(57) ABSTRACT

In the present disclosure, aspects of drug coatings and methods of making drug coated medical devices are disclosed. The methods of making a drug coated medical device may include applying a drug coating onto an exterior surface of a medical device for use within the body during interventional procedures and potentially residing after these procedures, including both implanted and non-implanted devices. The drug coating may include an anti-fibrosis drug, kinase inhibitor, phosphodiesterase inhibitor or combinations thereof.

### TECHNICAL FIELD

The present disclosure relates generally to coatings for medical devices and, more particularly, to drug-eluting stents and drug-coated balloon catheters.

### BACKGROUND

As background, drug and device combination products provide synergy of bare device function and pharmaceutical agent effect. Two examples of the combination devices include drug-coated medical devices, including drug-eluting stents and drug-coated balloon catheters.

It has become increasingly common to treat a variety of medical conditions by introducing a drug-coated medical device into the vascular system or other lumen within a human or veterinary patient such as the esophagus, trachea, colon, biliary tract, bronchial passages, sinus passages, nasal passages, renal arteries, or urinary tract. For example, medical devices, which may be coated and used for the treatment of vascular disease, include stents, stent grafts, catheters, balloon catheters, guide wires, cannulas and the like. While these medical devices initially appear successful, the benefits are often compromised by the occurrence of complications, such as late thrombosis, or recurrence of disease, such as stenosis (restenosis), after such treatment.

Combining drugs and medical devices is a complicated area of technology. It involves the usual formulation challenges, such as those of oral or injectable pharmaceuticals, together with the added challenge of maintaining drug adherence to the medical device until it reaches the target site and subsequently delivering the drug to the target tissues with the desired release and absorption kinetics. Furthermore, coatings must not impair functional performance such as burst pressure and compliance of balloons. The coating thickness must also be kept to a minimum, since a thick coating would increase the medical device's profile and lead to poor trackability and deliverability. These coatings generally contain almost no liquid chemicals, which typically are often used to stabilize drugs. Thus, formulations that are effective with ingestible tablets and capsules or with injectables might not work at all with coatings of a medical device.

Further, drug coatings must meet release profiles demanded by the nature of the underlying device. For example, balloons are typical in place within a patient transiently, requiring the ability to deliver the drug coating quickly. Balloons are also often fed into place by moving along the lumen of a blood vessel from an insertion point often distal to the area requiring the drug coating. Accordingly, the drug coating needs to be shielded or protected from premature delivery of the drug coating so that a maximal intended localized drug delivery is achieved at the intended area within the vessel. While balloons can provide a sustained release of drug, the drug coating lay needs to itself be deposited on the vessel wall safely and securely while the balloon is inflated in situ.

Stents also have particular requirements of a drug coating that vary considerably from a balloon. For example, a stent is typically placed permanently or semi-permanently within a subject and accordingly the requirement for immediate or rapid delivery of the coating to the vessel wall is less demanding. The length of presence of a stent further allows for a more dynamic release profile that can accommodate any period of time, thereby allowing the coating to remain on the surface of the stent and allow a timed release therefrom rather than needing a rapid transfer of the coating to the vessel. Stents also allow for more biodegradable polymers to be utilized in the drug coating, allowing for a slower and sustained drug release as the polymer slowly degrades from the surface of the stent.

Additionally, if a drug releases or elutes from the device too easily, it may be lost during device delivery before it can be deployed at the target site, or it may burst off the device during the initial phase of inflation and wash away before being pressed into contact with target tissue of a body lumen wall. If the drug adheres too strongly, the device may be withdrawn before the drug can be released and absorbed by tissues at the target tissues.

### SUMMARY

The reduction or elimination of restenosis after interventional procedures remains to be an unmet need for the development of the new generation of interventional devices. The implantation of conventional interventional devices, such as stents, balloon catheters and stent grafts, commonly causes vascular wall injury and endothelial denudation, followed by abnormal proliferation and migration of vascular smooth muscle cells (VSMCs), chronic inflammation and neointimal hyperplasia. In an effort to combat restenosis, drug eluting devices that locally deliver antiproliferative agents to blood vessels were developed.

Current anti-proliferative drug-coating therapies have demonstrated themselves to be effective to reduce restenosis through the inhibition of VSMC migration and proliferation. However, these current therapies act with a non-specific antiproliferative effect that also significantly inhibits endothelial cell (EC) growth, which results in delayed endothelium recovery and late stage thrombosis. As a result, there are needs for alternative anti-restenotic agents for drug coated devices, particularly for peripheral arterial diseases.

In some aspects, the present disclosure meets these needs by providing drug coatings and methods of coating medical devices, which include a therapeutic agent that does not inhibit endothelial cells or cause delayed endothelium recovery or late stage thrombosis. In some aspects, the therapeutic agent may be of one or more kinase inhibitors, which are used in the coatings of interventional devices for the prevention of restenosis. In certain aspects, the therapeutic agent may be one or more receptor tyrosine kinase inhibitors, which are used on the coatings of interventional devices for the prevention of restenosis.

A first aspect, either alone or in combination with any other aspect herein, concerns a medical device for delivering a therapeutic agent to a tissue, the medical device comprising: a coating layer overlying an exterior surface of the medical device, wherein the coating layer comprises a phosophodiesterase (PDE) inhibitor and/or a kinase inhibitor in combination with one or more excipients.

A second aspect, either alone or in combination with any other aspect herein, concerns the medical device of the first aspect, wherein the excipient comprises a biodurable polymer, a biodegradable polymer or a combination thereof.

A third aspect, either alone or in combination with any other aspect herein, concerns the medical device of the first aspect, wherein the PDE inhibitor is chosen from xanthine, aminophylline, sildenafil, tadalafil, vardenafil, udenafil, avanafil, dipyridamole, quinazolines, paraxanthine, papaverine, mesembrenone, rolipram, ibudilast, piclamilast, luteolin, drotaverine, roflumilast, apremilast, crisaborole, inamrinone, milrinone, enoximone, anagrelide, cilostazol, pimobendan, erythro-9-(2-hydroxy-3-nonyl)adenine), (2-[(3,4-dimethoxyphenyl)methyl]-7-[(1R)-1-hydroxyethyl]-4-phenylbutyl]-5-methyl-imidazo [5,1-f][1,2,4]triazin-4(1H)-one), oxindole, (9-(6-phenyl-2-oxohex-3-yl)-2-(3,4-dimethoxybenzyl)-purin-6-one), 3-isobutyl-1-methylxanthine, pentoxifylline, theobromine, and theophylline.

A fourth aspect, either alone or in combination with any other aspect herein, concerns the medical device of the first aspect, wherein the PDE inhibitor is tadalafil or sildenafil.

A fifth aspect, either alone or in combination with any other aspect herein, concerns the medical device of the first aspect, wherein the PDE inhibitor is in the form of a free base, a free acid, a crystal, or a salt.

A sixth aspect, either alone or in combination with any other aspect herein, concerns the medical device of the fifth aspect, wherein the salt is of a hydrochloride salt, a sodium salt, a sulfate salt, an acetate salt, a phosphate and/or diphosphate sale, a pamoate or hemi-pamoate salt, a lipophilic salt, a chloride salt, a potassium salt, a maleate salt, a calcium salt, a citrate salt, a mesylate salt, a nitrate salt, a tartrate salt, an aluminum salt, a stearic acid salt, a dioctyl sulfosuccinic acid or a gluconate salt.

A seventh aspect, either alone or in combination with any other aspect herein, concerns the medical device of the first aspect, wherein the kinase inhibitor is chosen from bosutinib, ceritinib, crizotinib, gefitinib, ruxolitinib, imatinib, axitinib, nilotinib, trametinib, afatinib, ibrutinib, cabozantinib, imatinib, lenvatinib, sunitinib, regorafenib, sorafenib, vandetanib, dasatinib, pazopanib, Y27632, CA3, verteporfin, VGLL4 peptide, nintedanib, avapritinib, abemaciclib, erdafitinib, fedratinib, palbociclib, and pemigatinib.

An eighth aspect, either alone or in combination with any other aspect herein, concerns the medical device of the first aspect, wherein the kinase inhibitor is sunitinib.

A ninth aspect, either alone or in combination with any other aspect herein, concerns the medical device of the first aspect, wherein the kinase inhibitor is in the form of a free base, a free acid, a crystal, or a salt.

A tenth aspect, either alone or in combination with any other aspect herein, concerns the medical device of the ninth aspect, wherein the salt is of a hydrochloride salt, a sodium salt, a sulfate salt, an acetate salt, a phosphate and/or diphosphate sale, a pamoate or hemi-pamoate salt, a lipophilic salt, a chloride salt, a potassium salt, a maleate salt, a calcium salt, a citrate salt, a mesylate salt, a nitrate salt, a tartrate salt, an aluminum salt, a stearic acid salt, a dioctyl sulfosuccinic acid or a gluconate salt.

An eleventh aspect, either alone or in combination with any other aspect herein, concerns the medical device of the second aspect, wherein the biodurable polymer is chosen from poly(vinylidene hexafluoropropylene) (PVDF-HFP), polyethylene terephthalate (PET), nylon 6,6, polyurethane (PU), polytetrafluoroethylene (PTFE), polyethylene, polysiloxanes (silicones) and poly(methylmethacrylate) (PMMA), and combinations thereof.

A twelfth aspect, either alone or in combination with any other aspect herein, concerns the medical device of the second aspect, wherein the biodurable polymer is poly(vinylidene hexafluoropropylene) (PVDF-HFP).

A thirteenth aspect, either alone or in combination with any other aspect herein, concerns the medical device of the second aspect, wherein a weight ratio of the biodurable polymer to the PDE inhibitor is from 1:1 to 10:1.

A fourteenth aspect, either alone or in combination with any other aspect herein, concerns the medical device of the second aspect, wherein a weight ratio of the biodurable polymer to the kinase inhibitor is from 1:1 to 10:1.

A fifteenth aspect, either alone or in combination with any other aspect herein, concerns the medical device of the second aspect, wherein the biodegradable polymer is chosen from a polylactic acid polymer, polycaprolactone (PCL), poly lactic-co-glycolic acid (PLGA), and poly(ethylene glycol) methyl ether-block-poly(lactide-co-glycolide) (PLGA-b-mPEG).

A sixteenth aspect, either alone or in combination with any other aspect herein, concerns the medical device of the second aspect, wherein the biodegradable polymer is PLGA.

A seventeenth aspect, either alone or in combination with any other aspect herein, concerns the medical device of the first aspect, wherein the medical device is chosen from a balloon catheter, a perfusion balloon catheter, an infusion catheter, a cutting balloon catheter, a scoring balloon catheter, a laser catheter, an atherectomy device, a debulking catheter, a stent, a filter, a stent graft, a covered stent, a patch, a wire, and a valve.

An eighteenth aspect, either alone or in combination with any other aspect herein, concerns the medical device of the first aspect, wherein the medical device is a stent or a stent graft.

A nineteenth aspect, either alone or in combination with any other aspect herein, concerns the medical device of the first aspect, wherein the medical device is a balloon catheter.

A twentieth aspect, either alone or in combination with any other aspect herein, concerns the medical device of the first aspect, wherein the coating layer comprises one or more additional excipients.

A twenty-first aspect, either alone or in combination with any other aspect herein, concerns the medical device of the twentieth aspect, wherein the one or more additional excipients are chosen from polylactic acid (PLLA,PDLA,PDLLA), polycaprolactone (PCL), sodium docusate, PLGA, PLGA-b-mPEG, polyglutamic acid, polyacrilic acid, hyaluronic acid, alginate, PVA, PVP, Pluronic (PEO—PPO-PEO), cellulose, CMC, HPC, starch, chitosan, human serum albumin (HSA), phospholipids, fatty acid, fatty acid esters, triglycerides, beeswax, cyclodextrin, Tween 20, Tween 80, TPGS, SLS, butylated hydroxytoluene, vitamin E, vitamin E succinate, tannic acid, polyethylene glycol, N-isopropylacrylamide, and sorbitol esters.

A twenty-second aspect, either alone or in combination with any other aspect herein, concerns the medical device of the first aspect, further comprising an antioxidant.

A twenty-third aspect, either alone or in combination with any other aspect herein, concerns the medical device of the twenty-second aspect, wherein the antioxidant is butylated hydroxytoluene.

A twenty-fourth aspect, either alone or in combination with any other aspect herein, concerns the medical device of the first aspect, wherein the tissue includes tissue of one of coronary vasculature, peripheral vasculature, cerebral vasculature, esophagus, airways, sinus, trachea, colon, biliary tract, urinary tract, prostate, and brain passages.

A twenty-fifth aspect, either alone or in combination with any other aspect herein, concerns a balloon catheter for delivering a therapeutic agent to a blood vessel, the balloon catheter comprising: an elongate member having a lumen and a distal end; an expandable balloon attached to the distal end of the elongate member and in fluid communication with the lumen; and a coating layer overlying an exterior surface of the balloon, the coating layer comprising a therapeutic agent and at least one of a biodegradable polymer and an excipient, wherein: the therapeutic agent comprises a PDE inhibitor, a kinase inhibitor, an anti-fibrotic drug, or mixtures thereof, the biodegradable polymer is chosen from a polylactic acid polymer, polycaprolactone (PCL), poly lactic-co-glycolic acid (PLGA), and poly(ethylene glycol) methyl ether-block-poly(lactide-co-glycolide) (PLGA-b-mPEG); and an excipient chosen from a fatty acid, a fatty acid ester, polylactic acid (PLLA,PDLA,PDLLA), polycaprolactone (PCL), sodium docusate, PLGA, PLGA-b-mPEG, polyglutamic acid, polyacrilic acid, hyaluronic acid, alginate, PVA, PVP, Pluronic (PEO-PPO-PEO), cellulose, CMC, HPC, starch, chitosan, human serum albumin (HSA), phospholipids, fatty acid, fatty acid esters, triglycerides, beeswax, cyclodextrin, Tween 20, Tween 80, TPGS, SLS, butylated hydroxytoluene, vitamin E, vitamin E succinate, tannic acid, polyethylene glycol, N-isopropylacrylamide, and sorbitol esters.

A twenty-sixth aspect, either alone or in combination with any other aspect herein, concerns the balloon catheter of the twenty-fifth aspect, wherein the PDE inhibitor is chosen from xanthine, aminophylline, sildenafil, tadalafil, vardenafil, udenafil, avanafil, dipyridamole, quinazoline, paraxanthine, papaverine, mesembrenone, rolipram, ibudilast, piclamilast, luteolin, drotaverine, roflumilast, apremilast, crisaborole, inamrinone, milrinone, enoximone, anagrelide, cilostazol, pimobendan, erythro-9-(2-hydroxy-3-nonyl) adenine), (2-[(3,4-dimethoxyphenyl)methyl]-7-[(1R)-1-hydroxyethyl]-4-phenylbutyl]-5-methyl-imidazo[5,1-f][1,2,4]triazin-4 (1H)-one), oxindole, (9-(6-phenyl-2-oxohex-3-yl)-2-(3,4-dimethoxybenzyl)-purin-6-one), 3-isobutyl-1-methylxanthine, pentoxifylline, theobromine, and theophylline.

A twenty-seventh aspect, either alone or in combination with any other aspect herein, concerns the balloon catheter of the twenty-fifth aspect, wherein the PDE inhibitor is tadalafil or sildenafil.

A twenty-eighth aspect, either alone or in combination with any other aspect herein, concerns the balloon catheter of the twenty-fifth aspect, wherein the PDE inhibitor is in the form of a free base, a crystal, a free acid or a salt.

A twenty-ninth aspect, either alone or in combination with any other aspect herein, concerns the balloon catheter of the twenty-eighth aspect, wherein the salt is of a hydrochloride salt, a sodium salt, a sulfate salt, an acetate salt, a phosphate and/or diphosphate sale, a pamoate or hemi-pamoate salt, a chloride salt, a potassium salt, a maleate salt, a calcium salt, a citrate salt, a mesylate salt, a nitrate salt, a tartrate salt, an aluminum salt, a stearic acid salt, a dioctyl sulfosuccinic acid or a gluconate salt.

A thirtieth aspect, either alone or in combination with any other aspect herein, concerns the balloon catheter of the twenty-fifth aspect, wherein the kinase inhibitor is chosen from bosutinib, ceritinib, crizotinib, gefitinib, ruxolitinib, imatinib, axitinib, nilotinib, trametinib, afatinib, ibrutinib, cabozantinib, imatinib, lenvatinib, sunitinib, regorafenib, sorafenib, vandetanib, dasatinib, pazopanib, Y27632, CA3, verteporfin, VGLL4 peptide, nintedanib, avapritinib, abemaciclib, erdafitinib, fedratinib, palbociclib, and pemigatinib.

A thirty-first aspect, either alone or in combination with any other aspect herein, concerns the balloon catheter of the twenty-fifth aspect, wherein the kinase inhibitor is sunitinib.

A thirty-second aspect, either alone or in combination with any other aspect herein, concerns the balloon catheter of the twenty-fifth aspect, wherein the kinase inhibitor is in the form of a free base, a free acid, a crystal, or a salt.

A thirty-third aspect, either alone or in combination with any other aspect herein, concerns the balloon catheter of the thirty-second aspect, wherein the salt is of a hydrochloride salt, a sodium salt, a sulfate salt, an acetate salt, a phosphate and/or diphosphate sale, a pamoate or hemi-pamoate salt, a lipophilic salt, a chloride salt, a potassium salt, a maleate salt, a calcium salt, a citrate salt, a mesylate salt, a nitrate salt, a tartrate salt, an aluminum salt, a stearic acid salt, a dioctyl sulfosuccinic acid or a gluconate salt.

A thirty-fourth aspect, either alone or in combination with any other aspect herein, concerns the balloon catheter of the twenty-fifth aspect, wherein the anti-fibrotic drug is chosen from triamciclone, tranilast, halofuginone, montelukast, zafirlukast, pirfenidone, nintedanib, and combinations thereof.

A thirty-fifth aspect, either alone or in combination with any other aspect herein, concerns the balloon catheter of the twenty-fifth aspect, wherein a weight ratio of the biodegradable polymer to the therapeutic agent is from 1:10 to 5:1.

A thirty-sixth aspect, either alone or in combination with any other aspect herein, concerns the balloon catheter of the twenty-fifth aspect, wherein the biodegradable polymer is PLGA.

A thirty-seventh aspect, either alone or in combination with any other aspect herein, concerns the balloon catheter of the twenty-fifth aspect, wherein the excipient is sodium docusate.

A thirty-eighth aspect, either alone or in combination with any other aspect herein, concerns the balloon catheter of the twenty-fifth aspect, further comprising an antioxidant.

A thirty-ninth aspect, either alone or in combination with any other aspect herein, concerns the balloon catheter of the thirty-eighth aspect, wherein the antioxidant is chosen from probucol, vitamin E, vitamin E succinate, butylated hydroxytoluene (BHT), ascorbic acid, beta carotene, lycopene, lutein, retinol, manganese, selenium, flavonoids, flavones, catechins, polyphenols, and/or zeaxanthin.

A fortieth aspect, either alone or in combination with any other aspect herein, concerns a stent, stent graft or other permanent or semi-permanent medical device for delivering a therapeutic agent to a blood vessel, comprising a device body and a drug coating thereon, wherein the drug coating comprises: a therapeutic agent and at least one of a biodurable polymer, and an excipient, wherein: the therapeutic agent comprises a PDE inhibitor, a kinase inhibitor, an anti-fibrotic drug, or mixtures thereof, the biodurable polymer is chosen from poly(vinylidene hexafluoropropylene) (PVDF-HFP), polyethylene terephthalate (PET), nylon 6,6, polyurethane (PU), polytetrafluoroethylene (PTFE), polyethylene, polysiloxanes (silicones) and poly(methylmethacrylate) (PMMA), and combinations thereof; and an excipient chosen from a fatty acid, a fatty acid ester, polylactic acid (PLLA,PDLA,PDLLA), polycaprolactone (PCL), sodium docusate, PLGA, PLGA-b-mPEG, polyglutamic acid, polyacrilic acid, hyaluronic acid, alginate, PVA, PVP, Pluronic (PEO—PPO-PEO), cellulose, CMC, HPC, starch, chitosan, human serum albumin (HSA), phospholipids, fatty acid, fatty acid esters, triglycerides, beeswax, cyclodextrin, Tween 20, Tween 80, TPGS, SLS, butylated hydroxytoluene, vitamin E, vitamin E succinate, and sorbitol esters.

A forty-first aspect, either alone or in combination with any other aspect herein, concerns the stent, stent graft or other permanent or semi-permanent medical device of the fortieth aspect, wherein the PDE inhibitor is chosen from xanthine, aminophylline, sildenafil, tadalafil, vardenafil, udenafil, avanafil, dipyridamole, quinazoline, paraxanthine, papaverine, mesembrenone, rolipram, ibudilast, piclamilast, luteolin, drotaverine, roflumilast, apremilast, crisaborole, inamrinone, milrinone, enoximone, anagrelide, cilostazol, pimobendan, erythro-9-(2-hydroxy-3-nonyl) adenine), (2-[(3,4-dimethoxyphenyl)methyl]-7-[(1R)-1-hydroxyethyl]-4-phenylbutyl]-5-methyl-imidazo [5,1-f][1,2,4]triazin-4 (1H)-one), oxindole, (9-(6-phenyl-2-oxohex-3-yl)-2-(3,4-dimethoxybenzyl)-purin-6-one), 3-isobutyl-1-methylxanthine, pentoxifylline, theobromine, and theophylline.

A forty-second aspect, either alone or in combination with any other aspect herein, concerns the stent, stent graft or other permanent or semi-permanent medical device of the fortieth aspect, wherein the PDE inhibitor is tadalafil or sildenafil.

A forty-third aspect, either alone or in combination with any other aspect herein, concerns the stent, stent graft or other permanent or semi-permanent medical device of the fortieth aspect, wherein the PDE inhibitor is in the form of a free base, a crystal, a free acid or a salt.

A forty-fourth aspect, either alone or in combination with any other aspect herein, concerns the stent, stent graft or other permanent or semi-permanent medical device of the forty-third aspect, wherein the salt is of a hydrochloride salt, a sodium salt, a sulfate salt, an acetate salt, a phosphate and/or diphosphate sale, a pamoate or hemi-pamoate salt, a chloride salt, a potassium salt, a maleate salt, a calcium salt, a citrate salt, a mesylate salt, a nitrate salt, a tartrate salt, an aluminum salt, a stearic acid salt, a dioctyl sulfosuccinic acid or a gluconate salt.

A forty-fifth aspect, either alone or in combination with any other aspect herein, concerns the stent, stent graft or other permanent or semi-permanent medical device of the fortieth aspect, wherein the kinase inhibitor is chosen from bosutinib, ceritinib, crizotinib, gefitinib, ruxolitinib, imatinib, axitinib, nilotinib, trametinib, afatinib, ibrutinib, cabozantinib, imatinib, lenvatinib, sunitinib, regorafenib, sorafenib, vandetanib, dasatinib, pazopanib, Y27632, CA3, verteporfin, VGLL4 peptide, nintedanib, avapritinib, abemaciclib, erdafitinib, fedratinib, palbociclib, and pemigatinib.

A forty-sixth aspect, either alone or in combination with any other aspect herein, concerns the stent, stent graft or other permanent or semi-permanent medical device of the fortieth aspect, wherein the kinase inhibitor is sunitinib.

A forty-seventh aspect, either alone or in combination with any other aspect herein, concerns the stent, stent graft or other permanent or semi-permanent medical device of the fortieth aspect, wherein the kinase inhibitor is in the form of a free base, a free acid, a crystal, or a salt.

A forty-eighth aspect, either alone or in combination with any other aspect herein, concerns the stent, stent graft or other permanent or semi-permanent medical device of the forty-seventh aspect, wherein the salt is of a hydrochloride salt, a sodium salt, a sulfate salt, an acetate salt, a phosphate and/or diphosphate sale, a pamoate or hemi-pamoate salt, a lipophilic salt, a chloride salt, a potassium salt, a maleate salt, a calcium salt, a citrate salt, a mesylate salt, a nitrate salt, a tartrate salt, an aluminum salt, a stearic acid salt, a dioctyl sulfosuccinic acid or a gluconate salt.

A forty-ninth aspect, either alone or in combination with any other aspect herein, concerns the stent, stent graft or other permanent or semi-permanent medical device of the fortieth aspect, wherein the anti-fibrotic drug is chosen from triamciclone, tranilast, halofuginone, montelukast, zafirlukast, pirfenidone, nintedanib, and combinations thereof.

A fiftieth aspect, either alone or in combination with any other aspect herein, concerns the stent, stent graft or other permanent or semi-permanent medical device of the fortieth aspect, wherein the biodurable polymer is PVDF-HFP.

A fifty-first aspect, either alone or in combination with any other aspect herein, concerns the stent, stent graft or other permanent or semi-permanent medical device of the fortieth aspect, wherein a weight ratio of the biodurable polymer to the therapeutic agent is from 1:1 to 10:1.

A fifty-second aspect, either alone or in combination with any other aspect herein, concerns the stent, stent graft or other permanent or semi-permanent medical device of the fortieth aspect, wherein the biodegradable polymer is PLGA.

A fifty-third aspect, either alone or in combination with any other aspect herein, concerns the stent, stent graft or other permanent or semi-permanent medical device of the fortieth aspect, wherein the excipient is sodium docusate.

A fifty-fourth aspect, either alone or in combination with any other aspect herein, concerns the stent, stent graft or other permanent or semi-permanent medical device of the fortieth aspect, further comprising an antioxidant.

A fifty-fifth aspect, either alone or in combination with any other aspect herein, concerns the stent, stent graft or other permanent or semi-permanent medical device of the fifty-fourth aspect, wherein the antioxidant is chosen from probucol, vitamin E, vitamin E succinate, butylated hydroxytoluene (BHT), ascorbic acid, beta carotene, lycopene, lutein, retinol, manganese, selenium, flavonoids, flavones, catechins, polyphenols, tannic acid, and/or zeaxanthin.

A kinase inhibitor, PDE inhibitor, and/or anti-fibrotic agent, for use in a method of relieving stenosis in a target tissue and/or preventing restenosis and/or late lumen loss of a body lumen, wherein the kinase inhibitor and/or anti-fibrotic gent is delivered to the target tissue by means of a medical device according to any one of aspects 1 to 55.

These and other features, aspects, and advantages of the present disclosure will become better understood with reference to the following description and the appended claims.

Additional features and advantages of the aspects described herein will be set forth in the detailed description which follows, and in part will be readily apparent to those skilled in the art from that description or recognized by practicing the aspects described herein, including the detailed description which follows, the claims, as well as the appended drawings.

### BRIEF DESCRIPTION OF THE DRAWINGS

It is to be understood that both the foregoing general description and the following detailed description describe various aspects and are intended to provide an overview or framework for understanding the nature and character of the claimed subject matter. The accompanying drawings are included to provide a further understanding of the various aspects, and are incorporated into and constitute a part of this specification. The drawings illustrate the various aspects described herein, and together with the description serve to explain the principles and operations of the claimed subject matter.

FIG. 1 is a schematic of an exemplary aspect of a medical device, particularly a balloon catheter, according to the present disclosure.

FIG. 2A is a cross-section of some aspect of the distal portion of the balloon catheter of FIG. 1, taken along line A-A, including a drug coating layer on an exterior surface of a balloon.

FIG. 2B and is a cross-section of some aspect of the distal portion of the balloon catheter of FIG. 1, taken along line A-A, including an intermediate layer between a exterior surface of the balloon and a drug coating layer.

FIG. 3 is a schematic of an exemplary aspect of a medical device, particularly a stent, according to the present disclosure.

FIG. 4 is a cross-section of some aspect of the distal portion of the balloon catheter of FIG. 3, taken along line A-A, including a drug coating layer on an exterior surface of a stent.

FIG. 5 is an optical microscopy image of Sample Stent 1.

FIG. 6 is an optical microscopy image of Sample Stent 2.

FIG. 7 is a cumulative release profile of Sample Stent 1 and Sample Stent 2.

FIG. 8 is an in vivo release profile and tissue PK graph over the time course up to 90 days after stent insertion.

FIG. 9 shows cross-section vessel views of Comparative A and C with Samples 1 and 2.

FIG. 10 shows in vivo pharmacokinetic data for sunitinib, tadalafil, colchicine, and roflumilast at 7 and 28 days after stent insertion.

FIG. 11 shows arterial cross-sections and stent endothelization at 60 days after stent insertion with drug coatings of colchicine, tadalafil, roflumlilast, sunitinib, and PVDF only.

FIG. 12 shows arterial cross-sections and stent endothelization at 90 days after stent insertion with drug coatings of colchicine, tadalafil, roflumlilast, sunitinib, and PVDF only.

FIG. 13 shows representative scanning electron microscopy images of PLGA/Sunitnib malate microparticle prepared using O/W emulsion evaporation method showing spherical morphology (Top Panel) at low magnification and (Bottom Panel) at high magnification.

### DETAILED DESCRIPTION

Specific aspects of the present application will now be described. These aspects are provided so that this disclosure will be thorough and complete and will fully convey the scope of the subject matter to those skilled in the art.

Except where stated otherwise, all molecular weights herein are reported in Daltons (g/mol). Molecular weights of polymeric materials are reported as weight-average molecular weights.

As used herein, the interchangeable terms “coating” and “layer” refer to material that is applied, or that has been applied, onto a surface or a portion of a surface of a substrate using any customary application or deposition method such as vapor deposition, spray coating, dip coating, lamination, bonding, micropatterning, molding, painting, spin coating, sputtering, immersion coating, plasma-assisted deposition, or vacuum evaporation, for example.

The terms “coated” and “applied” as verbs may be used interchangeably herein. Except where stated otherwise, a reference to a “substrate coated with a certain material” or the like is equivalent to a “substrate to which a certain material has been applied” to a surface or a portion of a surface of the substrate using any customary application or deposition method such as vapor deposition, spray coating, dip coating, painting, spin coating, sputtering, immersion coating, plasma-assisted deposition, or vacuum evaporation, for example.

### Drug Coating

In some aspects, the present disclosure concerns one or more drug coatings for medical devices and the uses the same. The medical device may include angioplasty balloons, catheters, guide wires, balloons, filters, stents, stent grafts, vascular grafts, aneurysm filling coils, meshes, artificial heart valves, pace maker leads, ports, needles, clips and all other devices with drug coating. In some aspects, the presently-disclosed drug coatings may be applied over an exterior surface of an expandable medical device, including, as non-limiting examples, balloon catheters and stents. Exemplary methods for preparing the medical devices and the coatings thereon are set forth herein as well as exemplary data from expandable medical devices including the drug coatings described herein. In some aspects, the drug coating may be of the therapeutic agent itself. In other aspects, the drug coating may include a therapeutic agent and an additive. In further aspects, the drug coating may of the therapeutic agent and two or more additives. In some aspects, the additive may include a polymer.

As stated previously, current commercial drug coated devices, while able to effectively reduce restenosis through the inhibition of VSMC migration and proliferation, act with sufficient non-specificity that also causes inhibition of endothelial cell (EC) growth and/or proliferation, delay in endothelium recovery and late stage thrombosis. Collectively, these non-specific and non-desirable effects increase the long-term risk of mortality in patients.

In other aspects, the present disclosure provides a drug coating for medical devices, which may include an anti-fibrosis drug, a kinase inhibitor, a phosphodiesterase inhibitor, or combinations thereof. One further non-specific effect that can be seen with some current drug-coated devices includes the stimulated production of fibrin, which can lead to fibrosis. One downside of current coated angioplasty balloons is that the contained drugs act in a non-discriminatory anti-proliferative manner and can become dislodged and move to other parts of the body. Thus, unintended consequences can result as the non-specific anti-proliferative drug residue moves downstream in the body from an inserted device. For example, dislodged drug can migrate to the lung and cause fibrotic scarring therein. By including an anti-fibrosis drug in the coating of a device as set forth herein, the potential to stimulate fibrin production or to induce fibrotic tissue is protected against.

In some aspects, the presently-described drug coatings may allow for the effective and efficient delivery of therapeutic agents, drugs, or bioactive materials directly into a localized tissue area during or following a medical procedure, so as to treat or prevent vascular and nonvascular diseases such as restenosis. The presently-described drug coatings may allow for the release of therapeutic agent in an effective and efficient manner at the desired target location, where the therapeutic agent can permeate the target tissue to treat disease, for example, to relieve stenosis and prevent restenosis and late lumen loss of a body lumen. In some further aspects, the presently-described drug coatings may allow for the release of therapeutic agent in an effective and efficient manner for the treatment of pulmonary fibrosis. Moreover, presently-described drug coatings may allow for effective treatment without significantly inhibiting endothelial cells (EC).

In some aspects, the drug coating contains at least one therapeutic agent that is present at a desired concentration density thereon. In some aspects, the concentration density of the at least one therapeutic agent in the drug coating may be from about 0.1 μg/mm² to about 10 μg/mm², including about 0.2, 0.25, 0.3, 0.35, 0.4, 0.45, 0.5, 0.55, 0.6, 0/65, 0.7, 0.75, 0.8, 0.85, 0.9, 0.95, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, and 9.9 μg/mm². In other aspects, the concentration density of the at least one therapeutic agent in the drug coating may be from about 0.1 μg/mm² to about 8 μg/mm², from about 0.1 μg/mm² to about 6 μg/mm², from about 0.1 μg/mm² to about 4 μg/mm², from about 0.1 μg/mm² to about 2 μg/mm², from about 0.1 μg/mm² to about 1 μg/mm², from about 1 μg/mm² to about 10 μg/mm², from about 1 μg/mm² to about 8 μg/mm², from about 1 μg/mm² to about 6 μg/mm², from about 1 μg/mm² to about 4 μg/mm², from about 1 μg/mm² to about 2 μg/mm², from about 2 μg/mm² to about 10 μg/mm², from about 2 μg/mm² to about 8 μg/mm², from about 2 μg/mm² to about 6 μg/mm², from about 2 μg/mm² to about 4 μg/mm², from about 4 μg/mm² to about 10 μg/mm², from about 4 μg/mm² to about 8 μg/mm², from about 4 μg/mm² to about 6 μg/mm², from about 6 μg/mm² to about 10 μg/mm², from about 6 μg/mm² to about 8 μg/mm², or from about 8 μg/mm² to about 10 μg/mm². In some aspects, the concentration density of the at least one therapeutic agent in the drug coating may be from about 0.5 μg/mm² to about 5 μg/mm².

In some aspects, as set forth herein, the drug-coating may include a polymer or two or more polymers. In some aspects, the ratio by weight of the polymer to the therapeutic agent in the drug coating may be from about 1:1 to about 10:1, from about 1:1 to about 9:1, from about 1:1 to about 8:1, from about 1:1 to about 7:1, from about 1:1 to about 6:1, from about 1:1 to about 5:1, from about 1:1 to about 4:1, from about 1:1 to about 3:1, from about 1:1 to about 2:1, from about 2:1 to about 10:1, from about 2:1 to about 9:1, from about 2:1 to about 8:1, from about 2:1 to about 7:1, from about 2:1 to about 6:1, from about 2:1 to about 5:1, from about 2:1 to about 4:1, from about 2:1 to about 3:1, from about 3:1 to about 10:1, from about 3:1 to about 9:1, from about 3:1 to about 8:1, from about 3:1 to about 7:1, from about 3:1 to about 6:1, from about 3:1 to about 5:1, from about 3:1 to about 4:1, from about 4:1 to about 10:1, from about 4:1 to about 9:1, from about 4:1 to about 8:1, from about 4:1 to about 7:1, from about 4:1 to about 6:1, from about 4:1 to about 5:1, from about 5:1 to about 10:1, from about 5:1 to about 9:1, from about 5:1 to about 8:1, from about 5:1 to about 7:1, from about 5:1 to about 6:1, from about 6:1 to about 10:1, from about 6:1 to about 9:1, from about 6:1 to about 8:1, from about 6:1 to about 7:1, from about 7:1 to about 10:1, from about 7:1 to about 9:1, from about 7:1 to about 8:1, from about 8:1 to about 10:1, from about 8:1 to about 9:1, or from about 9:1 to about 10:1. If the ratio (by weight) of the polymer to the therapeutic agent is too low, then drug may release prematurely, and if the ratio is too high, then drug may not elute quickly enough or be absorbed by tissue when deployed at the target site. For example, a low ratio may lead to a faster release and a high ratio may lead to a slower release.

In some aspects, the drug-coating may include a biodurable polymer. As set forth herein, a biodurable polymer may include a polymer that is well-tolerated and/or non-reactive when contacted to a subject or immune-reactive cells thereof and is resistant to erosion and/or enzymatic degradation and/or dissolution within the subject or the circulatory system thereof. (See, e.g., Nathanael et al. Polymer 2020, 12, 3061; doi: 10.3390/polym 12123061; “Polymers for Vascular and Urogenital Applications” Shalaby et al. eds. CRC Pres, 2017; and “Concise Encyclopedia of Biomedical Polymers and Polymeric Biomaterials” Mishra ed. CRC Press 2017). By way of example, biodurable polymers include polyethylene terephthalate (PET), nylon 6,6, polyurethane (PU), polytetrafluoroethylene (PTFE), polyethylene (PE, low density and high density and ultra-high molecular weight, UHMW), polysiloxanes (silicones) and poly(methylmethacrylate) (PMMA) and Poly(vinylidene fluoride-co-hexafluoropropylene) (PVDF-HFP). In some aspects, the biodurable polymer may be PVDF-HFP.

In other aspects, the drug-coating may include a biodegradable polymer. As set forth herein, a biodegradable polymer may include a polymer that is well-tolerated and/or non-reactive when contacted to a subject or immune-reactive cells thereof and is prone to to erosion and/or enzymatic degradation and/or dissolution within the subject or the circulatory system thereof over a course of time. (See, e.g., Nathanael et al. Polymer 2020, 12, 3061; doi: 10.3390/polym12123061; “Polymers for Vascular and Urogenital Applications” Shalaby et al. eds. CRC Pres, 2017; and “Concise Encyclopedia of Biomedical Polymers and Polymeric Biomaterials” Mishra ed. CRC Press 2017). Biodegradable polymers allow for the reduction or elimination of incomplete drug release. Examples of biodegradable polymers include polylactic acid polymers (PLA, PLLA, PDLA, PDLLA), polycaprolactone (PCL), poly lactic-co-glycolic Acid (PLGA), and poly(ethylene glycol) methyl ether-block-poly(lactide-co-glycolide) (PLGA-b-mPEG).

In some aspects, the drug coating is configured for the underlying medical device. For example, in some aspects the medical device is a stent and in other aspects, the medical device is a balloon. Accordingly, the drug coating may be tailored to the characteristics of a transient device that is fed along the vessel wall and the drug coating can be tailored for a permanent or semi-permanent device that can release the drug directly from the surface as the device resides within the vessel.

In some aspects, the present disclosure concerns a drug coating for a balloon or inflatable device intended to inflate and exert pressure from the inside to the out of a vessel. Due to the obstructive nature to the circulation caused by the inflation, these devices are deployed only for shortened periods of time. Due to such constraints, it will be apparent that the drug coating needs to be configured for rapid delivery to the vessel wall. In some aspects, the balloon can deliver the drug coating in a manner that either releases the coating to the vessel wall or allows for rapid absorbance by the vessel wall. In some aspects, the drug coating can be configured with an excipient and/or drug solvation to encourage transfer to the interior of the vessel wall. In some aspects, excipients may include an excipient as set forth herein. In other aspects, the excipient may include a polyethylene glycol (PEG), urea, polylactic acid (PLA), poly glycolic acid (PGA), poly lactic-co-glycolic acid (PLGA), shellac, dimethyl sulfoxide (DMSO), polysorbate, sodium docusate, sorbitol, butyryl trihexyl citrate (BTHC), N-isopropylacryla mide (P-NIPAAm), or combinations thereof. In some aspects, the drug coating may be of a particular formulation of the therapeutic agent, such as a crystal and/or microparticle thereof or a salt and/or microparticle thereof. In some aspects, the salt may be a malate salt. In some aspects, the drug coating may include a polymer, such as a biodegradable and/or bioerodible polymer as set forth herein or combinations thereof. In some aspects, the drug coating may include PLA, PGA and/or PLGA. In some aspects, the drug coating may be in the form of a free base. In some aspects, the drug coating may be of an excipient, a salt of the therapeutic agent and a biodegradable and/or bioerodible polymer.

In other aspects, the drug coating is configured for a stent, a stent graft or other longer residing medical devices, such as a perfusion balloon catheter, an infusion catheter, a cutting balloon catheter, a scoring balloon catheter, a laser catheter, an atherectomy device, a debulking catheter, a filter, a stent graft, a covered stent, a patch, a wire, and a valve. Due to the permanent or semi-permanent nature of the stent, the drug coating need not transfer the active agent with the same urgency and can be configured to remain on the surface or outer regions of the stent to provide a drug release profile required by the condition. For example, combining the therapeutic agent with a biodurable polymer, a biodegradable and/or bioerodible polymer can embed the therapeutic and allow for sustained and/or delayed release as the polymer erodes. Such polymers may include PLA, PGA, PLGA, polyvinylidene fluoride (PVD or PVDF), polyvinylidene fluoride-hexafluoropropylene (PVDF-HFP or PVD-HFP), poly(n-butyl methacrylate (PBMA), polystyrene-b-polyisobutylene-b-polystyrene (SIBS) or combinations thereof. In some aspects the drug coating may include the therapeutic in one or more formulations to provide a preferred release profile such as with different loadings within a polymer, different particle sizes and combinations salts/crystal/free base forms of the therapeutic.

Many aspects of the present disclosure may be particularly useful for treating vascular disease and for reducing stenosis and late luminal loss, or are useful in the manufacture of devices for that purpose or in methods of treating that class of diseases. Though the examples set forth herein are described only with respect to stents and balloon catheters, it should be understood that, in addition to stents and balloon catheters, other medical devices, particularly other expandable medical devices, may be coated with a drug coating that comprises a therapeutic agent and an additive, such as described previously with respect to stents and balloon catheters. Such other medical devices include, without limitation, stent grafts, scoring balloon catheters, and recanalization catheters.

### Therapeutic Agent

In some aspects, the drug-coating of the medical device may include at least one therapeutic agent. A therapeutic may include a small molecule chemical compound in an uncharged or neutral state, an anion thereof, a cation thereof, a salt thereof, a derivative thereof and/or a crystal thereof or crystalline form thereof. In some aspects, the drug coating of the medical device may include a therapeutic agent and at least one additive. In some aspects, the drug coating may include an anti-fibrosis drug, a kinase inhibitor, or a combination thereof, which may be viable targets for the treatment of restenosis with improved specificity and less adverse effects compared with non-specific anti-proliferative drugs.

As used herein, “derivative” may refer to a chemically or biologically modified version of a chemical compound that is structurally similar to a parent compound and (actually or theoretically) derivable from that parent compound (for example, dexamethasone). A derivative may or may not have different chemical or physical properties of the parent compound. For example, the derivative may be more hydrophilic or it may have altered reactivity as compared to the parent compound. Derivatization (i.e., modification) may involve substitution of one or more moieties within the molecule (e.g., a change in functional group). For example, a hydrogen may be substituted with a halogen, such as fluorine or chlorine, or a hydroxyl group (—OH) may be replaced with a carboxylic acid moiety (—COOH). The term “derivative” may also include conjugates, and prodrugs of a parent compound (i.e., chemically modified derivatives which can be converted into the original compound under physiological conditions). For example, the prodrug may be an inactive form of an active agent. Under physiological conditions, the prodrug may be converted into the active form of the compound, such as through phase I and/or II of a metabolic pathway. Prodrugs may be formed, for example, by replacing one or two hydrogen atoms on nitrogen atoms by an acyl group (acyl prodrugs) or a carbamate group (carbamate prodrugs). More detailed information relating to prodrugs is found, for example, in Fleisher et al., Advanced Drug Delivery Reviews 19 (1996) 115; Design of Prodrugs, H. Bundgaard (ed.), Elsevier, 1985; or H. Bundgaard, Drugs of the Future 16 (1991) 443. The term “derivative” is also used to describe all solvates, for example hydrates or adducts (e.g., adducts with alcohols), active metabolites, and salts of the parent compound. The type of salt that may be prepared depends on the nature of the moieties within the compound. For example, acidic groups, for example carboxylic acid groups, can form alkali metal salts or alkaline earth metal salts (e.g., sodium salts, potassium salts, magnesium salts and calcium salts, as well as salts with physiologically tolerable quaternary ammonium ions and acid addition salts with ammonia and physiologically tolerable organic amines such as triethylamine, ethanolamine or tris-(2-hydroxyethyl)amine). Basic groups can form acid addition salts, for example with inorganic acids such as hydrochloric acid, sulfuric acid or phosphoric acid, or with organic carboxylic acids and sulfonic acids such as acetic acid, citric acid, benzoic acid, maleic acid, fumaric acid, tartaric acid, methanesulfonic acid or p-toluenesulfonic acid. In some aspects, organic acids can include fatty acids such as stearic acid and/or dioctyl sulfosuccinic acid. In some aspects, organic acids can include those with biological activity, such as oleanolic acid, betulinic acid, ursolic acid, and/or vaprolic acid. In other aspects, the organic acids can include those with antioxidant properties, such as ascorbic acid, tannic acid and vitamin E succinate. In some aspects, the organic acids can include pamoic acid. Compounds which simultaneously contain a basic group and an acidic group, for example a carboxyl group in addition to basic nitrogen atoms, can be present as zwitterions. Salts can be obtained by customary methods known to those skilled in the art, for example by combining a compound with an inorganic or organic acid or base in a solvent or diluent, or from other salts by cation exchange or anion exchange.

As used herein, “analog” or “analogue” may refer to a chemical compound that is structurally similar to another but differs slightly in composition (as in the replacement of one atom by an atom of a different element or in the presence of a particular functional group), but may or may not be derivable from the parent compound. A “derivative” may differ from an “analog” or “analogue” in that a parent compound may be the starting material to generate a “derivative,” whereas the parent compound may not necessarily be used as the starting material to generate an “analog.”

In some aspects of the present disclosure, the therapeutic agent or substance may include drugs or biologically active materials. The drugs can be of various physical states, e.g., molecular distribution, crystal forms, cluster forms, or combinations thereof. Examples of drugs that may demonstrate the specific anti-proliferative actions and/or lack the non-specific inhibition of endothelial cell growth and/or proliferation or endothelialization may include phosphodiesterase inhibitor drugs and/or anti-fibrosis drugs and/or kinase inhibitors and/or tyrosine kinase inhibitors and/or receptor tyrosine kinase inhibitors. Further examples of drugs may include one or more of bosutinib, ceritinib, crizotinib, gefitinib, ruxolitinib, imatinib, axitinib, nilotinib, trametinib, afatinib, ibrutinib, cabozantinib, imatinib, lenvatinib, sunitinib, regorafenib, sorafenib, vandetanib, dasatinib, pazopanib, triamciclone, tranilast, halofuginone, montelukast, zafirlukast, pirfenidone, Y27632, CA3, verteporfin, VGLL4 peptide, nintedanib, avapritinib, abemaciclib, erdafitinib, fedratinib, nilotinib, nintendanib, palbociclib, pemigatinib, xanthines, aminophylline, sildenafil, tadalafil, vardenafil, udenafil, avanafil, dipyridamole, quinazoline, paraxanthine, papaverine, mesembrenone, rolipram, ibudilast, piclamilast, luteolin, drotaverine, roflumilast, apremilast, crisaborole, inamrinone, milrinone, enoximone, anagrelide, cilostazol, pimobendan, erythro-9-(2-hydroxy-3-nonyl) adenine), (2-[(3,4-dimethoxyphenyl)methyl]-7-[(1R)-1-hydroxyethyl]-4-phenylbutyl]-5-methyl-imidazo [5,1-f][1,2,4]triazin-4 (1H)-one), oxindole, (9-(6-phenyl-2-oxohex-3-yl)-2-(3,4-dimethoxybenzyl)-purin-6-one), 3-isobutyl-1-methylxanthine, pentoxifylline, theobromine, and theophylline. In certain aspects, these drugs may be suitable for use in a coating on an expandable medical device used to treat tissue of the vasculature.

In some aspects, the therapeutic agent may be include protein kinase inhibitors, which may also be referred to as multi-targeted tyrosine kinase inhibitors (MTK), such as cabozantib, imatinib, lenvatinib, sunitinib, regorafenib, sorafenib, vandetanib, dasatinib, and pazopanib. Protein kinases are a large family of enzymes that regulate biological responses including cell proliferation and inflammation through an enzymatic cascade of phosphorylation events. Tyrosine kinases refer to the tyrosine amino acid(s) involved in the phosphorylation in a particular enzyme or receptor and resulting enzymatic activity, with serine and threonine amino acids being the other main amino acids for kinase activation and signal transduction. One example is the receptor protein tyrosine kinase platelet-derived growth factor receptor (PDGFR), which, upon bound with the endogenous platelet-derived growth factor (PDGF or platelet-derived growth hormone PDGH), may initiate the cascade the results in vascular smooth muscle cell (VSMC) migration and proliferation. Up-regulated expression of PDGF and PDGFR has been reported in the injured vascular tissues. In addition, vascular endothelial growth factor (VEGF) and its tyrosine kinase receptor VEGFR has also been demonstrated to be highly involved in the pathological progress of restenosis. Examples of PDGFR and/or VEGFR inhibitors, including inhibitors of enzymes downstream thereof, include imatinib, nintedanib, sorafenib, sunitinib, and pazopanib, ROCK inhibitor (Y27632), YAP/TAZ inhibitor (CA3 and verteporfin), YAP/TAZ-TEAD interaction inhibitor (verteporfin, VGLL4 peptide), SRC inhibitor (dasatinib). Further, a kinase modulator derived from a bioactive product may be included, such as resveratrol, quercetin, curcumin, chrysin, myricetin, luteolin, apigenin, anthrocyanin, genistein, epigallocatechin gallate, fisetin, astxanthin, tetrahydrocurcumin, and/or combinations thereof.

Consequently, inhibition of PDGF and/or VEGF and/or activation of the tyrosine kinase receptors thereof (i.e. VEGFR and/or PDGFR) offers a more selective approach than general cytotoxic agents in preventing the formation of neointimal hyperplasia without equally suppressing both vascular smooth muscle cells (VSMC) and/or normal cells. Moreover, tyrosine kinase inhibitors, such as sunitinib, target proliferative smooth muscle cells in a more selective way than drugs currently used for drug coated interventional devices. In some aspects, the local delivery of kinase inhibitors using the interventional devices described herein, followed by a sustained drug release, may allow for inhibited restenosis without causing systemic toxicity. Additionally, in general, kinase inhibitors offer good chemically stability as they do not readily degrade during typical storage conditions for the medical devices set forth herein.

Kinase inhibitors are typically weak bases protonated under physiological conditions. As a result, tyrosine kinase inhibitors have a higher water solubility. In some aspects, the drug coating may include a therapeutic agent having a water solubility of from about 0.1 mg/mL to about 50 mg/mL, from about 0.1 mg/mL to about 8 mg/mL, from about 0.1 mg/mL to about 6 mg/mL, from about 0.1 mg/mL to about 4 mg/mL, from about 0.1 mg/mL to about 2 mg/mL, from about 2 mg/mL to about 10 mg/mL, from about 2 mg/mL to about 8 mg/mL, from about 2 mg/mL to about 6 mg/mL, from about 2 mg/mL to about 4 mg/mL, from about 4 mg/mL to about 10 mg/mL, from about 4 mg/mL to about 8 mg/mL, from about 4 mg/mL to about 6 mg/mL, from about 6 mg/mL to about 10 mg/mL, from about 6 mg/mL to about 8 mg/mL, from about 8 mg/mL to about 10 mg/mL, from about 6 mg/mL to about 20 mg/mL, from about 8 mg/mL to about 25 mg/mL, from about 10 mg/mL to about 25 mg/mL, from about 10 mg/mL to about 30 mg/mL, from about 15 mg/mL to about 35 mg/mL, from about 20 mg/mL to about 40 mg/mL, from about 30 mg/mL to about 45 mg/mL, from about 30 mg/mL to about 50 mg/mL, from about 35 mg/mL to about 50 mg/mL, from about 40 mg/mL to about 50 mg/mL, from about 45 mg/mL to about 50 mg/mL, from about 10 mg/mL to about 50 mg/mL, or from about 1 mg/mL to about 50 mg/mL. For example, the water solubility of sunitinib malate is about 25 mg/mL. In some aspects, while high solubility may present a challenge for sustained release formulations, the use of lipophilic excipients within the coating matrix can slow down or inhibit the drug dissolution.

In some aspects, the kinase inhibitor may be coated on the medical device directly as a free base or free acid. In other aspects, the kinase inhibitor may be protonated or in a salt form, such as a hydrochloride salt, a sodium salt, a sulfate salt, an acetate salt, a phosphate and/or diphosphate sale, a pamoate or hemi-pamoate salt, a chloride salt, a potassium salt, a maleate salt, a calcium salt, a citrate salt, a mesylate salt, a nitrate salt, a tartrate salt, an aluminum salt, or a gluconate salt.

In conventional methods, systemic administration of a kinase inhibitor may require a relatively high dose, presumably causing severe side effects. In some present aspects, the concentration density of the kinase inhibitor in the drug coating may be from about 0.1 μg/mm² to about 10 μg/mm², from about 0.1 μg/mm² to about 8 μg/mm², from about 0.1 μg/mm² to about 6 μg/mm², from about 0.1 μg/mm² to about 4 μg/mm², from about 0.1 μg/mm² to about 2 μg/mm², from about 0.1 μg/mm² to about 1 μg/mm², from about 1 μg/mm² to about 10 μg/mm², from about 1 μg/mm² to about 8 μg/mm², from about 1 μg/mm² to about 6 μg/mm², from about 1 μg/mm² to about 4 μg/mm², from about 1 μg/mm² to about 2 μg/mm², from about 2 μg/mm² to about 10 μg/mm², from about 2 μg/mm² to about 8 μg/mm², from about 2 μg/mm² to about 6 μg/mm², from about 2 μg/mm² to about 4 μg/mm², from about 4 μg/mm² to about 10 μg/mm², from about 4 μg/mm² to about 8 μg/mm², from about 4 μg/mm² to about 6 μg/mm², from about 6 μg/mm² to about 10 μg/mm², from about 6 μg/mm² to about 8 μg/mm², or from about 8 μg/mm² to about 10 μg/mm². In some aspects, the concentration density of the at least one therapeutic agent in the drug coating may be from about 0.5 μg/mm² to about 5 μg/mm².

In some aspects, the therapeutic agent may be an anti-fibrotic drug. Anti-fibrosis pharmacological mechanisms of action include the inhibition and/or reduction in localized inflammation, and reduction and/or inhibition of the formation of fibrous tissue growth factors. Anti-fibrotic drugs may include, for example, triamciclone, tranilast, halofuginone, montelukast, zafirlukast, pirfenidone and nintedanib. For example, therapeutic agents such as pirfenidone and nintedanib may slow the progression of scar tissue build up.

In some aspects, the therapeutic agent of the drug-coating may be of at least one tyrosine kinase inhibitor, at least one receptor tyrosine kinase inhibitor, at least one anti-fibrotic agent or any combination thereof. In some aspects, the therapeutic agent may include at least one of cabozantinib, imatinib, lenvatinib, sunitinib, regorafenib, sorafenib, vandetanib, dasatinib, pazopanib triamciclone, tranilast, halofuginone, montelukast, zafirlukast, pirfenidone, nintedanib, ROCK inhibitor (Y27632), YAP/TAZ inhibitor (CA3 and verteporfin), YAP/TAZ-TEAD interaction inhibitor (verteporfin, VGLL4 peptide), SRC inhibitor (dasatinib), or a salt thereof, or a crystal or crystalline form thereof or a derivative thereof.

In some aspects, the therapeutic agent can be a phosphodiesterase (PDE) inhibitor. A PDEinhibitor refers to a class of pharmaceutical that are characterized by their activity in inhibiting PDE enzymatic activity. PDE enzymes are a class of enzymes that catalyze the breaking of a phosphodiester bond which are in cyclic nucleotide compounds such as cyclic adenosine monophosphate (cAMP) and cyclic guanosine monophosphate (cGMP). There are multiple isoforms of PDE with each particular protein receiving a numerical identifier. In some aspects, application of a PDE inhibitor can prevent the cleavage of CAMP and/or cGMP. In some aspects, application of a PDE inhibitor can increase levels of CAMP and/or cGMP. Accordingly, a PDE inhibitor may offer a more selective approach in preventing the formation of neointimal hyperplasia without negatively suppressing other surrounding cells. In some aspects, the local delivery of PDE inhibitors using the interventional devices described herein, followed by a sustained drug release, may allow for inhibited restenosis without causing systemic toxicity.

Some PDE inhibitors or at least salts thereof are typically weak bases protonated under physiological conditions and accoridngly have a higher water solubility. In some aspects, the drug coating may include a therapeutic agent having a water solubility of from about 0.1 mg/mL to about 25 mg/mL, from about 0.1 mg/mL to about 8 mg/mL, from about 0.1 mg/mL to about 6 mg/mL, from about 0.1 mg/mL to about 4 mg/mL, from about 0.1 mg/mL to about 2 mg/mL, from about 2 mg/mL to about 10 mg/mL, from about 2 mg/mL to about 8 mg/mL, from about 2 mg/mL to about 6 mg/mL, from about 2 mg/mL to about 4 mg/mL, from about 4 mg/mL to about 10 mg/mL, from about 4 mg/mL to about 8 mg/mL, from about 4 mg/mL to about 6 mg/mL, from about 6 mg/mL to about 10 mg/mL, from about 6 mg/mL to about 8 mg/mL, from about 8 mg/mL to about 10 mg/mL, from about 6 mg/mL to about 15 mg/mL, from about 8 mg/mL to about 15 mg/mL, from about 10 mg/mL to about 20 mg/mL, from about 10 mg/mL to about 25 mg/mL, from about 15 mg/mL to about 20 mg/mL, from about 15 mg/mL to about 25 mg/mL, from about 20 mg/mL to about 25 mg/mL, from about 10 mg/mL to about 25 mg/mL, or from about 1 mg/mL to about 25 mg/mL.

In some aspects, the therapeutic agent can include a non-selective PDE inhibitor, wherein the therapeutic may inhibit two or more PDE enzymes. Examples of non-selective PDE inhibitors can include xanthines, caffeine, aminophylline, 3-isobutyl-1-methylxanthine, pentoxifylline, theobromine, and theophylline.

In some aspects, the PDE inhibitor may be a selective PDEinhibitor wherein the therapeutic agent preferentially target one isoform of PDE enzyme. For example, in some aspects, the PDE inhibitor may be a PDE2 inhibitor, such as erythro-9-(2-hydroxy-3-nonyl) adenine), (2-[(3,4-dimethoxyphenyl)methyl]-7-[(1R)-1-hydroxyethyl]-4-phenylbutyl]-5-methyl-imidazo[5,1-f][1,2,4]triazin-4 (1H)-one), oxindole, and/or 9-(6-phenyl-2-oxohex-3-yl)-2-(3,4-dimethoxybenzyl)-purin-6-one. In some aspects, the PDE inhibitor is a PDE3 inhibitor, such as inamrinone, milrinone, enoximone, anagrelide, cilostazol, and/or pimobendan, In some aspects, the PDE inhibitor is a PDE4 inhibitor, such as mesembrenone, rolipram, ibudilast, piclamilast, luteolin, drotaverine, roflumilast, apremilast, and/or crisaborole. In some aspects, the PDE inhibitor is a PDE5 inhibitor such as sildenafil, tadalafil, vardenafil, udenafil, avanafil, and/or dipyridamole. In some aspects, the PDE inhibitor is a PDE 7 inhibitor such as quinazoline. In some aspects, the PDE inhibitor is a PDE9 inhibitor such as paraxanthine. In some aspects, the PDE inhibitor is a PDE10 inhibiot such as papaverine.

In some present aspects, the concentration density of the PDE inhibitor in the drug coating may be from about 0.1 μg/mm² to about 10 μg/mm², from about 0.1 μg/mm² to about 8 μg/mm², from about 0.1 μg/mm² to about 6 μg/mm², from about 0.1 μg/mm² to about 4 μg/mm², from about 0.1 μg/mm² to about 2 μg/mm², from about 0.1 μg/mm² to about 1 μg/mm², from about 1 μg/mm² to about 10 μg/mm², from about 1 μg/mm² to about 8 μg/mm², from about 1 μg/mm² to about 6 μg/mm², from about 1 μg/mm² to about 4 μg/mm², from about 1 μg/mm² to about 2 μg/mm², from about 2 μg/mm² to about 10 μg/mm², from about 2 μg/mm² to about 8 μg/mm², from about 2 μg/mm² to about 6 μg/mm², from about 2 μg/mm² to about 4 μg/mm², from about 4 μg/mm² to about 10 μg/mm², from about 4 μg/mm² to about 8 μg/mm², from about 4 μg/mm² to about 6 μg/mm², from about 6 μg/mm² to about 10 μg/mm², from about 6 μg/mm² to about 8 μg/mm², or from about 8 μg/mm² to about 10 μg/mm². In some aspects, the concentration density of the at least one therapeutic agent in the drug coating may be from about 0.5 μg/mm² to about 5 μg/mm².

In some aspects, the therapeutic agent is a combination of a kinase inhibitor and an anti-fibrotic drug. In some aspects, the therapeutic selected includes a combination of a kinase inhibitor and a PDE inhibitor. In some aspects, the therapeutic selected includes a combination of an anti-fibrotic and a PDE inhibitor. In some aspects, the therapeutic selected includes at least one kinase inhibitor and at least one anti-fibrotic drug and at least one PDE inhibitor.

In some aspects, the therapeutic agent can be applied to an outer surface of a medical device or a coating thereon. In some aspects, the therapeutic may be directly applied. In other aspects, the therapeutic may be applied after combining with a coating solvent. Those skilled in the art will appreciate that a combination of approached to coating the therapeutic may also be utilized to coat the medical device.

In some aspects, other therapeutic compounds can be included with the drug-coating of the present disclosure. Such other drugs may include, without limitation, glucocorticoids (e.g., cortisol, betamethasone), hirudin, angiopeptin, aspirin, growth factors, antisense agents, anti-cancer agents, anti-proliferative agents, oligonucleotides, and, more generally, anti-platelet agents, anti-coagulant agents, anti-mitotic agents, antioxidants, anti-metabolite agents, anti-chemotactic, and anti-inflammatory agents. Also useful in some aspects of the present disclosure are polynucleotides, antisense, RNAi, or siRNA, for example, that inhibit inflammation and/or smooth muscle cell or fibroblast proliferation, contractility, or mobility, including lipid nanoparticles encasing the same. Anti-platelet agents can include drugs such as aspirin and dipyridamole. Aspirin is classified as an analgesic, antipyretic, anti-inflammatory and anti-platelet drug. Dipyridamole is a drug similar to aspirin in that it has anti-platelet characteristics. Dipyridamole is also classified as a coronary vasodilator. Anti-coagulant agents for use in some aspects of the present disclosure can include drugs such as heparin, protamine, hirudin and tick anticoagulant protein. Anti-oxidant agents can include probucol, vitamin E, vitamin E succinate, butylated hydroxytoluene (BHT), ascorbic acid, beta carotene, lycopene, lutein, retinol, manganese, selenium, flavonoids, flavones, catechins, polyphenols, and/or zeaxanthin. Anti-proliferative agents can include drugs such as amlodipine and doxazosin. Anti-mitotic agents and anti-metabolite agents that can be used in some aspects of the present disclosure include drugs such as methotrexate, azathioprine, vincristine, adriamycin, and mutamycin. Antibiotic agents for use in some aspects of the present disclosure include penicillin, cefoxitin, oxacillin, tobramycin, and gentamicin. Suitable antioxidants for use in some aspects of the present disclosure include probucol. Additionally, genes or nucleic acids, or portions thereof can be used as the therapeutic agent in aspects of the present disclosure. Photosensitizing agents for photodynamic or radiation therapy, including various porphyrin compounds such as porfimer, for example, are also useful as drugs in aspects of the present disclosure.

A combination of drugs can also be used in some aspects of the present disclosure. Some of the combinations have additional effects and/or super-additional effects because they have a different mechanisms. In some aspects, the additional effects may be advantageous for use in the drug coatings described herein. For example, in some aspects, because of the additional effects, the dose of the drug can be reduced. In some aspects, combinations of therapeutic agents may reduce complications from using a high dose of the therapeutic agent.

In some aspects of the present disclosure, the therapeutic agent is rapidly released from the drug-coating after the medical device is brought into contact with tissue and is readily absorbed. For example, certain aspects of devices of the present disclosure include drug coated expandable medical devices that deliver a proliferative pharmaceutical to vascular tissue through brief, direct pressure contact at high drug concentration during balloon angioplasty. The therapeutic agent is preferentially retained in target tissue at the delivery site, where it inhibits hyperplasia and restenosis yet allows endothelialization. In these aspects, coating formulations of the present disclosure not only facilitate rapid release of drug from the balloon surface and transfer of drug into target tissues during deployment, but also prevent drug from diffusing away from the device during transit through tortuous arterial anatomy prior to reaching the target site and from exploding off the device during the initial phase of balloon inflation, before the drug coating is pressed into direct contact with the surface of the vessel wall.

### Excipients

In some aspects, the drug-coating may be of a therapeutic agent and one or more additives. In some aspects, the additive may be an excipient. In addition to the therapeutic agent or combination of therapeutic agents, the drug coating according to some aspects may include at least one excipient. In one aspects, the drug coating may include multiple excipients, for example, two, three, four or more excipients. Such combinations of excipients may be useful for purposes of the present disclosure.

Selection of the excipient or combination of excipients may be based on the therapeutic agent, coating solvent, and/or coating solvents used. As explained subsequently in more detail, in some aspects, the excipient or combination of excipients can be mixed with the therapeutic agent(s) or with the therapeutic agent(s) and coating solvent (or a mixture of coating solvents) to form a coating mixture, which is coated onto the exterior surface of a medical device. Alternatively or additionally, some aspects of the present disclosure may include applying the excipients to the exterior surface of the medical device separately from the therapeutic agent dissolved in the coating solvent. In some aspects, the excipient or combination of excipients may be applied to the medical device before the therapeutic agent(s) and/or before the therapeutic agent(s) dissolved in the coating solvent. In some aspects, the excipient or combination of excipients may be applied to the medical device after the therapeutic agent(s) and/or therapeutic agent(s) dissolved in the coating solvent. Without being bound by theory, the chosen excipient or combination of excipients, when mixed with the therapeutic agent, coating solvent, and/or coating solvents, may form a coating mixture that adheres to the medical device such that the coating particles do not fall off during handling and interventional procedure. Alternatively or additionally, the chosen excipient or combination of excipients, when applied prior to or subsequently after the therapeutic agent, coating solvent, and/or coating solvents, should adhere to the medical device such that the coating particles do not fall off during handling and interventional procedure.

The relative amount of the therapeutic agent and the one or more excipients in the drug coating may vary depending on applicable circumstances. The optimal amount of the one or more excipients can depend upon, for example, the particular therapeutic agent and other excipients selected, the critical micelle concentration of the surface modifier if it forms micelles, the hydrophilic-lipophilic-balance (HLB) of the excipients, the one or more excipients' octonol-water partition coefficient (P), the melting point of the excipients, the water solubility of the excipients and/or therapeutic agent, the surface tension of water solutions of the surface modifier, etc. Other considerations will further inform the choice of specific proportions of the excipients. These considerations include the degree of bioacceptability of the excipients and the desired dosage of therapeutic agent to be provided.

In some aspects, the excipient may include a polymer. In some aspects, the polymer may be an anionic polymer. Examples of anionic polymers include polyglutamic acid or any block polymers containing this segment, polyacrylic acid or any block polymers containing this segment, polymethylacrylic acid or any block polymers containing this segment, polystyrene sulfonate or any block polymers containing this segment, heparin, hyaluronic acid, and alginate. Without being bound by theory, because of the cationic nature of the therapeutic agent, such as sunitinib malate, a drug coating including an anionic polymer may allow for the therapeutic agent to be retained for sustained drug release.

In further aspects, the excipient may be a biodurable polymer. A biodurable polymer may refer to a polymer that is well-tolerated and/or erosion or enzyme resistant when placed within a human body, including within the lumen of a blood vessel. Biodurable polymers include polyethylene terephthalate (PET), nylon 6,6, polyurethane (PU), polytetrafluoroethylene (PTFE), polyethylene (PE, low density and high density and ultra-high molecular weight, UHMW), polysiloxanes (silicones) and poly(methylmethacrylate) (PMMA)N-isopropylacryla mide (P-NIPAAm) and Poly(vinylidene fluoride-co-hexafluoropropylene) (PVDF-HFP). In some aspects, the excipient may be PVDF-HFP. Without being bound by theory, utilizing a biodurbale polymer allows for the reduction or elimination of incomplete drug release. In further aspects, the excipient may be a biodegradable polymer. A biodegradable polymer may include a polymer that is well tolerated and degradable over a period of time when introduced within a human body, including within a lumen of a blood vessel. Examples of biodegradable polymers include polylactic acid polymers (PLA, PLLA, PDLA, PDLLA), polycaprolactone (PCL), poly lactic-co-glycolic Acid (PLGA), polyethylene glycol (PEG), and poly(ethylene glycol) methyl ether-block-poly(lactide-co-glycolide) (PLGA-b-mPEG).

In some aspects, the weight ratio of the polymer to the therapeutic agent may be from about 0.5:1 to about 8:1, including about 1:1, 2:1, 3:1, 4:1, 5:1, 6:1, and 7:1. In some aspects, the ratio is of 1:1. In other aspects, the ratio is of 2:1. In some aspects, the ratio is of about 1:1 to about 8:1 or from about 1:1 to about 7:1 or from about 1:1 to about 6:1 or from about 1:1 to about 5:1 or from about 1:1 to about 4:1 or from about 1:1 to about 3:1 or from about 1:1 to about 2:1. In some aspects, the ratio is of about 2:1 to about 8:1. In some aspects, the ratio is of about 3:1. In other aspects, the ratio is of about 3:1 to about 5:1 or to about 8:1, including from about 3:1 to about 4:1 from about 3:1 to about 5:1 from about 3:1 to about 6:1 from about 3:1 to about 7:1. In some aspects, the ratio is of about 4:1 to about 8:1. In some aspects the ratio is of about 5:1 to about 8:1 from about 5:1 to about 7:1, from about 5:1 to about 6:1, from about 6:1 to about 8:1, from about 6:1 to about 7:1, or from about 7:1 to about 8:1.

Suitable excipients that can be used in some aspects of the present disclosure include, without limitation, those already described or listed herein, organic and inorganic pharmaceutical excipients, natural products and derivatives thereof (such as sugars, vitamins, amino acids, peptides, proteins, fatty acid esters, and fatty acids), surfactants (anionic, cationic, non-ionic, and ionic), and mixtures thereof. The following list of excipients useful in the present disclosure is provided for exemplary purposes only and is not intended to be comprehensive. Many other excipients may be useful for purposes of the present disclosure, such as polyglutamic acid, polyacrylic acid, hyaluronic acid, alginate, PVA, PVP, Pluronic (PEO—PPO-PEO), cellulose, PEG, P-NIPAAm, CMC, HPC, starch, chitosan, human serum albumin (HSA), phospholipids, fatty acid, fatty acid esters, triglycerides, beeswax, cyclodextrin, polysorbates, polyethylene glycol, polyvinylpyrrolidone (PVP) and aliphatic polyesters.

In some aspects, the excipients may have a drug affinity part. The drug affinity part includes an affinity to the therapeutic agent by hydrogen bonding and/or van der Waals interactions. For example, the drug affinity part of the excipients may bind the excipients to an anti-fibrosis drug, kinase inhibitor, tyrosine kinase inhibitor, PDE inhibitor, or combinations thereof. The excipients of aspects of the present disclosure may include a hydrophilic part. As is well known in the art, the terms “hydrophilic” and “hydrophobic” are relative terms. To function as an excipient in exemplary aspects of the present disclosure, the excipient may be of a compound that includes polar or charged hydrophilic moieties as well as non-polar hydrophobic (lipophilic) moieties. The hydrophilic part or moiety can accelerate diffusion and increase permeation of the therapeutic agent into tissue. The hydrophilic part of the excipient may facilitate rapid movement or transfer of therapeutic agent off the surface of the expandable medical device during deployment at the target site by preventing hydrophobic drug molecules from amassing together and to the device, thereby increasing drug solubility in interstitial spaces, and/or accelerating drug passage through polar head groups to the lipid bilayer of cell membranes of target tissues.

An empirical parameter commonly used to characterize the relative hydrophilicity and hydrophobicity of an excipient is the hydrophilic-lipophilic balance (“HLB” value). Excipients with lower HLB values are more hydrophobic, and have greater solubility in oils, while surfactants with higher HLB values are more hydrophilic, and have greater solubility in aqueous solutions. Using HLB values as a rough guide, hydrophilic excipients are generally considered to be those compounds having an HLB value greater than about 10, as well as anionic, cationic, or zwitterionic compounds for which the HLB scale is not generally applicable. Similarly, hydrophobic excipients are compounds having an HLB value less than about 10. The HLB values of excipients in certain aspects are in the range of from 0.0 to 40. In certain aspects of the present disclosure, a higher HLB value may be preferred, since increased hydrophilicity may facilitate release of therapeutic agent from the surface of the device. In one aspects, the HLB of the excipient is higher than 10. In another aspect, the excipient HLB may be higher than 14. Alternatively, excipient having lower HLB may be preferred when used to prevent drug loss prior to device deployment at the target site, for example in a separate top coat over a drug layer that has a very hydrophilic additive. It should be understood that the HLB value of an excipient is merely a rough guide generally used to enable formulation of industrial, pharmaceutical and cosmetic emulsions, for example. Keeping these inherent difficulties in mind, and using HLB values as a guide, excipients may be identified that have suitable hydrophilicity or hydrophobicity for use in some aspects of the present disclosure, as described herein.

An empirical parameter commonly used in medicinal chemistry to characterize the relative hydrophilicity and hydrophobicity of pharmaceutical compounds is the partition coefficient, P, the ratio of concentrations of un-ionized compound in the two phases of a mixture of two immiscible solvents, usually octanol and water, such that P=([solute]octanol/[solute]water). Compounds with higher log Ps are more hydrophobic, while compounds with lower log Ps are more hydrophilic. Lipinski's rule suggests that pharmaceutical compounds having log P<5 are typically more membrane permeable. For purposes of certain aspects of the present disclosure, it is preferable that the excipient has log P less than log P of the drug to be formulated (as an example, log P of paclitaxel is 7.4). A greater log P difference between the therapeutic agent and the excipient can facilitate phase separation of the therapeutic agent. For example, if log P of the excipient is much lower than log P of the drug, the excipient may accelerate the release of therapeutic agent in an aqueous environment from the surface of a device to which the therapeutic agent might otherwise tightly adhere, thereby accelerating drug delivery to tissue during brief deployment at the site of intervention. In certain aspects of the present disclosure, log P of the excipient is negative. In other aspects, log P of the excipient is less than log P of the therapeutic agent. While a compound's octanol-water partition coefficient P or log P is useful as a measurement of relative hydrophilicity and hydrophobicity, it is merely a rough guide that may be useful in defining suitable excipients for use in some aspects of the present disclosure.

Exemplary excipients for use in some aspects of the present disclosure may include chemical compounds with one or more hydroxyl, amino, carbonyl, carboxyl, acid, amide or ester moieties. Hydrophilic chemical compounds with one or more hydroxyl, amino, carbonyl, carboxyl, acid, amide or ester moieties having a molecular weight less than 5,000 to 10,000 are preferred in certain aspects. In other aspects, molecular weight of the excipient with one or more hydroxyl, amino, carbonyl, carboxyl, acid, amide, or ester moieties is preferably less than 1000 to 5,000, or more preferably less than 750 to 1,000, or most preferably less than 750. In these aspects, the molecular weight of the excipient may be preferred to be less than that of the therapeutic agent to be delivered.

The excipients according to some aspects may include amino alcohols, alcohols, amines, acids, amides and hydroxyl acids in both cyclo and linear aliphatic and aromatic groups. Examples are L-ascorbic acid and its salt, D-glucoascorbic acid and its salt, tromethamine, triethanolamine, diethanolamine, meglumine, glucamine, amine alcohols, glucoheptonic acid, glucomic acid, hydroxyl ketone, hydroxyl lactone, gluconolactone, glucoheptonolactone, glucooctanoic lactone, gulonic acid lactone, mannoic lactone, ribonic acid lactone, lactobionic acid, glucosamine, glutamic acid, benzyl alcohol, benzoic acid, hydroxybenzoic acid, propyl 4-hydroxybenzoate, lysine acetate salt, gentisic acid, lactobionic acid, lactitol, sorbitol, glucitol, sugar phosphates, glucopyranose phosphate, sugar sulphates, sugar alcohols, sinapic acid, vanillic acid, vanillin, methyl paraben, propyl paraben, xylitol, 2-ethoxyethanol, sugars, galactose, glucose, ribose, mannose, xylose, sucrose, lactose, maltose, arabinose, lyxose, fructose, cyclodextrin, (2-hydroxypropyl)-cyclodextrin, acetaminophen, ibuprofen, retinoic acid, lysine acetate, gentisic acid, catechin, catechin gallate, tiletamine, ketamine, propofol, lactic acids, acetic acid, salts of any organic acid and amine described above, polyglycidol, glycerol, multiglycerols, galactitol, di(ethylene glycol), tri (ethylene glycol), tetra(ethylene glycol), penta (ethylene glycol), di(propylene glycol), tri (propylene glycol), tetra(propylene glycol, and penta (propylene glycol), and combinations thereof. Some of the chemical compounds with one or more hydroxyl, amine, carbonyl, carboxyl, amide or ester moieties described herein are very stable under heating, survive an ethylene oxide sterilization process, and/or do not react with the therapeutic agent during sterilization.

In some aspects, the excipients may include amino acids and salts thereof. For example, the excipient may be one or more of alanine, arginine, asparagines, aspartic acid, cysteine, cystine, glutamic acid, glutamine, glycine, histidine, proline, isoleucine, leucine, lysine, methionine, phenylalanine, serine, threonine, tryptophan, tyrosine, valine, and derivatives thereof are. Certain amino acids, in their zwitterionic form and/or in a salt form with a monovalent or multivalent ion, have polar groups, relatively high octanol-water partition coefficients, and are useful in some aspects of the present disclosure. In the context of the present disclosure “low-solubility amino acid” refers to amino acid having a solubility in unbuffered water of less than about 4% (40 mg/ml). These include cystine, tyrosine, tryptophan, leucine, isoleucine, phenylalanine, asparagine, aspartic acid, glutamic acid, and methionine.

Amino acid dimers, sugar-conjugates, and other derivatives may also be useful. Through simple reactions well known in the art hydrophilic molecules may be joined to hydrophobic amino acids, or hydrophobic molecules to hydrophilic amino acids, to make additional excipients useful in some aspects of the present disclosure. Catecholamines, such as dopamine, levodopa, carbidopa, and DOPA, may also useful as excipients.

In some aspects, the excipients may be liquid additives. One or more liquid excipients may be can be used in the medical device coating to improve the integrity of the coating. Without being bound by theory, a liquid excipient can improve the compatibility of the therapeutic agent in the coating mixture. The liquid excipients used in some aspects of the present disclosure is not a solvent. The solvents such as ethanol, methanol, dimethylsulfoxide, and acetone, will be evaporated after the coating is dried. In other words, the solvent will not stay in the coating after the coating is dried. In contrast, the liquid excipients in some aspects of the present disclosure may remain in the coating after the coating is dried. The liquid excipient is liquid or semi-liquid at room temperature and one atmosphere pressure. The liquid excipient may form a gel at room temperature. The liquid excipient may include a hydrophilic part and a drug affinity part, wherein the drug affinity part is at least one of a hydrophobic part, a part that has an affinity to the therapeutic agent by hydrogen bonding, and a part that has an affinity to the therapeutic agent by van der Waals interactions. In some aspects, the liquid excipient may be a non-ionic surfactant. Examples of liquid excipients include PEG-fatty acids and esters, PEG-oil transesterification products, polyglyceryl fatty acids and esters, Propylene glycol fatty acid esters, PEG sorbitan fatty acid esters, and PEG alkyl ethers as mentioned above. Some examples of a liquid excipient are Tween 80, Tween 81, Tween 20, Tween 40, Tween 60, Solutol HS 15, Cremophor RH40, PEG, N-PIAAm, and Cremophor EL&ELP.

In some aspects, the excipient may be a surfactant; a chemical compound with one or more hydroxyl, amine, carbonyl, carboxyl, amides or ester moieties; or both. Exemplary surfactants may be chosen from PEG fatty esters, PEG omega-3 fatty esters and alcohols, glycerol fatty esters, sorbitan fatty esters, PEG glyceryl fatty esters, PEG sorbitan fatty esters, sugar fatty esters, PEG sugar esters, Tween 20, Tween 40, Tween 60, p-isononylphenoxypolyglycidol, PEG laurate, PEG oleate, PEG stearate, PEG glyceryl laurate, PEG glyceryl oleate, PEG glyceryl stearate, polyglyceryl laurate, polyglyceryl oleate, polyglyceryl myristate, polyglyceryl palmitate, polyglyceryl-6 laurate, polyglyceryl-6 oleate, polyglyceryl-6 myristate, polyglyceryl-6 palmitate, polyglyceryl-10 laurate, polyglyceryl-10 oleate, polyglyceryl-10 myristate, polyglyceryl-10 palmitate, PEG sorbitan monolaurate, PEG sorbitan monolaurate, PEG sorbitan monooleate, PEG sorbitan stearate, PEG oleyl ether, PEG laurayl ether, Tween 20, Tween 40, Tween 60, Tween 80, sodium docusate, octoxynol, monoxynol, tyloxapol, sucrose monopalmitate, sucrose monolaurate, decanoyl-N-methylglucamide, n-decyl-β-D-glucopyranoside, n-decyl-β-D-maltopyranoside, n-dodecyl-β-D-glucopyranoside, n-dodecyl-β-D-maltoside, heptanoyl-N-methylglucamide, n-heptyl-β-D-glucopyranoside, n-heptyl-β-D-thioglucoside, n-hexyl-β-D-glucopyranoside, nonanoyl-N-methylglucamide, n-nonyl-β-D-glucopyranoside, octanoyl-N-methylglucamide, n-octyl-β-D-glucopyranoside, octyl-β-D-thioglucopyranoside and their derivatives.

In some aspects, one or more of a surfactant or a small water-soluble molecule (the chemical compounds with one or more hydroxyl, amine, carbonyl, carboxyl, amides or ester moieties) with the therapeutic agent are in certain cases superior to only utilizing the therapeutic agent and a single excipient. By incorporating the one or more additional excipients, aspects of the drug coating may have increased stability during transit and rapid drug release when pressed against tissues of the lumen wall at the target site of therapeutic intervention when compared to some formulations comprising the therapeutic agent and only one excipient. Furthermore, the miscibility and compatibility of the therapeutic agent with the excipient or the drug coating with the medical device, generally, is improved by the presence of the one or more additional excipients. For example, a surfactant may allow for improved coating uniformity and integrity.

In one aspect, the drug coating may be of multiple excipients, with one excipient being more hydrophilic than one or more of the other excipients. In another aspects, the drug coating may be of multiple excipients, with one excipient being of a different structure from that of one or more of the other excipients. In another aspect, the drug coating may include multiple excipients, with one excipient possessing a different HLB value from that of one or more of the other excipients. In yet another aspect, the drug coating may include multiple excipients, with one excipient possessing a different Log P value from that of one or more of the other excipients.

Some aspects of the present disclosure may include a mixture of at least two additional excipients, for example, a combination of one or more surfactants and one or more chemical compound with one or more hydroxyl, amine, carbonyl, carboxyl, amides or ester moieties. For example, therapeutic agents may bind to extremely water-soluble small molecules more poorly than they do surfactants, which can lead to suboptimal coating uniformity and integrity. Some surfactants, when used in some aspects of the present disclosure, adhere so strongly to the therapeutic agents and the surface of the medical device that the therapeutic agent is not able to rapidly release from the surface of the medical device at the target site. On the other hand, some water-soluble small molecules (with one or more hydroxyl, amine, carbonyl, carboxyl, amides or ester moieties) adhere so poorly to the medical device that they release therapeutic agents before it reaches the target site, for example, into serum during the transit of a coated balloon catheter to the site targeted for intervention. By incorporating a mixture of multiple excipients, some aspects of the drug coating may have improved properties to a formulation comprising only one excipient.

In some aspects, the one or more additional excipients may include an antioxidant. An antioxidant is a molecule capable of slowing or preventing the oxidation of other molecules. Oxidation reactions can produce free radicals and/or peroxides, which start chain reactions and may cause degradation of sensitive therapeutic agents, for example of sunitinib and its derivatives. Antioxidants terminate these chain reactions by removing free radicals and/or peroxides, and they further inhibit oxidation of the active agent by being oxidized themselves. Antioxidants are used as the one or more additional excipients in certain aspects to prevent or slow the oxidation of the therapeutic agents in the coatings for medical devices. Antioxidants are a type of free radical scavengers. The antioxidant may be used alone or in combination with other additional excipients in certain aspects and may prevent degradation of the active therapeutic agent during sterilization or storage prior to use. Some representative examples of antioxidants that may be used in the drug coatings of the present disclosure include, without limitation, oligomeric or polymeric proanthocyanidins, polyphenols, polyphosphates, polyazomethine, high sulfate agar oligomers, chitooligosaccharides obtained by partial chitosan hydrolysis, polyfunctional oligomeric thioethers with sterically hindered phenols, hindered amines such as, without limitation, p-phenylene diamine, trimethyl dihydroquinolones, and alkylated diphenyl amines, substituted phenolic compounds with one or more bulky functional groups (hindered phenols) such as tertiary butyl, arylamines, phosphites, hydroxylamines, and benzofuranones. Also, aromatic amines such as p-phenylenediamine, diphenylamine, and

N,N′ disubstituted p-phenylene diamines may be utilized as free radical scavengers. Other examples include, without limitation, butylated hydroxytoluene (“BHT”), butylated hydroxyanisole (“BHA”), L-ascorbate (Vitamin C), Vitamin E, tannic, acid, herbal rosemary, sage extracts, glutathione, resveratrol, ethoxyquin, rosmanol, isorosmanol, rosmaridiphenol, propyl gallate, gallic acid, tannic acid, caffeic acid, p-coumeric acid, p-hydroxy benzoic acid, astaxanthin, ferulic acid, dehydrozingerone, chlorogenic acid, ellagic acid, propyl paraben, sinapic acid, daidzin, glycitin, genistin, daidzein, glycitein, genistein, isoflavones, and tertbutylhydroquinone. Examples of some phosphites include di(stearyl) pentaerythritol diphosphite, tris(2,4-di-tert.butyl phenyl)phosphite, dilauryl thiodipropionate and bis(2,4-di-tert.butyl phenyl) pentaerythritol diphosphite. Some examples, without limitation, of hindered phenols include octadecyl-3,5,di-tert.butyl-4-hydroxy cinnamate, tetrakis-methylene-3-(3′,5′-di-tert.butyl-4-hydroxyphenyl) propionate methane 2,5-di-tert-butylhydroquinone, ionol, pyrogallol, retinol, and octadecyl-3-(3,5-di-tert.butyl-4-hydroxyphenyl) propionate. An antioxidant may include glutathione, lipoic acid, melatonin, tocopherols, tocotrienols, thiols, Beta-carotene, retinoic acid, cryptoxanthin, 2,6-di-tert-butylphenol, propyl gallate, catechin, catechin gallate, and quercetin. Preferable antioxidants are butylated hydroxytoluene (BHT) and butylated hydroxyanisole (BHA).

In some aspects, the excipient may be present in an amount relative to the amount of one or more therapeutics. In some aspects, the excipient to therapeutic ratio may be of about 1:20 to about 10:1, including 1:15, 1:10, 1:5, 1:3, 1:2, 1:1, 2:18, 2:16, 2:14, 2:12, 2:1, 3:18, 3:15, 3:10, 3:9, 3:7, 3:5, 3:1, 4:1, 5:1, 6:1, 7:1, 8:1, 9:1, and 10:1.

### Coating Solvents

Solvents for preparing of the drug coating, which are referred to herein as “coating solvents” are used to dissolve the therapeutic agent and the additive. The dissolved therapeutic agent and additive in coating solvent together make up a “coating mixture,” which is coated onto the medical device.

The coating solvent may be any solvent or combination of solvents that are suitable to dissolve the selected therapeutic agent. Coating solvents may include, as examples, any combination of one or more of the following: water; alkanes such as pentane, cyclopentane, hexane, cyclohexane, heptane, and octane; aromatic solvents such as benzene, toluene, and xylene; alcohols such as methanol, ethanol, 2,2,2-trifluroethanol, propanol, and isopropanol, iso-butanol, n-butanol, tert-butanol, diethylamide, ethylene glycol monoethyl ether, trascutol, and benzyl alcohol; ethers such as dioxane, dimethyl ether, ethyl ether, diethyl ether, di-n-propyl ether, diisopropyl ether, t-butyl methyl ether, petroleum ether, and tetrahydrofuran; esters/acetates such as methyl acetate, ethyl acetate, isobutyl acetate, i-propyl acetate, and n-butyl acetate; ketones such as acetone, acetonitrile, diethyl ketone, cyclohexanone, and methyl ethyl ketones, methyl isobutyl ketone; chlorinated hydrocarbons such as chloroform, dichloromethane, ethylene dichloride; carbon tetrachloride, and chlorobenzene; dioxane; tetrahydrofuran; dimethylformamide; acetonitrile; dimethylsulfoxide; 1,6-dioxane; N,N-Dimethylacetamide (DMA); diethylene glycol; diglyme; 1,2-dimethoxy ethane; hexamethylphosphoramide; and mixtures such as water/ethanol, water/acetone, water/methanol, water/tetrahydrofuran.

The therapeutic agent and/or the additive or additives may be dispersed in, solubilized, or otherwise mixed in the coating solvent. The weight percent of therapeutic agent, the additive, and, optionally, one or more additional additives in the coating solvent may be in the range of from about 0.1% to about 80% by weight, or from about 0.1% to about 60%, from about 0.1% to about 40%, from about 0.1% to about 20%, from about 0.1% to about 1%, from about 1% to about 80%, from about 1% to about 60%, from about 1% to about 40%, from about 1% to about 20%, from about 20% to about 80%, from about 20% to about 60%, from about 20% to about 40%, from about 40% to about 80%, from about 40% to about 60%, or from about 60% to about 80% by weight.

In methods of preparing the drug coated medical device, particularly, for example, a balloon catheter or a stent, a coating solution or suspension including at least one coating solvent, a therapeutic agent, and optionally one or more additional additives is prepared. In some aspects, the therapeutic agent, the coating solvent, the additive, and optionally one or more additional additives may be combined to produce a coating mixture.

In aspects where the coating solution includes at least one coating solvent, a therapeutic agent, and optionally one or more additional additives, the content of the therapeutic agent in the coating solution can be from about 0.05% to about 50%, from about 0.05% to about 40%, from about 0.05% to about 30%, from about 0.05% to about 20%, from about 0.05% to about 10%, from about 0.05% to about 1%, from about 1% to about 50%, from about 1% to about 40%, from about 1% to about 30%, from about 1% to about 20%, from about 1% to about 10%, from about 10% to about 50%, from about 10% to about 40%, from about 10% to about 30%, from about 10% to about 20%, from about 20% to about 50%, from about 20% to about 40%, from about 20% to about 30%, from about 30% to about 50%, from about 30% to about 40%, or from about 40% to about 50% by weight, based on the total weight of the solution. The amount of coating solvent used depends on the coating process and viscosity, as the amount of solvent may affect the uniformity of the drug coating even though the coating solvent will be evaporated.

In aspects where the coating solution comprises at least one coating solvent, a therapeutic agent, an additive, and optionally one or more additional additives, the content of the therapeutic agent in the coating solution can be from about 0.1% to about 50%, from about 0.1% to about 40%, from about 0.1% to about 30%, from about 0.1% to about 20%, from about 0.1% to about 10%, from about 0.1% to about 1%, from about 1% to about 50%, from about 1% to about 40%, from about 1% to about 30%, from about 1% to about 20%, from about 1% to about 10%, from about 10% to about 50%, from about 10% to about 40%, from about 10% to about 30%, from about 10% to about 20%, from about 20% to about 50%, from about 20% to about 40%, from about 20% to about 30%, from about 30% to about 50%, from about 30% to about 40%, from about 40% to about 50%, by weight based on the total weight of the solution. The amount of coating solvent used depends on the coating process and viscosity, as the amount of solvent may affect the uniformity of the drug coating even though the coating solvent will be evaporated. The content of the additive in the coating solution can be from about 0.5% to about 50%, from about 0.5% to about 40%, from about 0.5% to about 30%, from about 0.5% to about 20%, from about 0.5% to about 10%, from about 0.5% to about 1%, from about 1% to about 50%, from about 1% to about 40%, from about 1% to about 30%, from about 1% to about 20%, from about 1% to about 10%, from about 10% to about 50%, from about 10% to about 40%, from about 10% to about 30%, from about 10% to about 20%, from about 20% to about 50%, from about 20% to about 40%, from about 20% to about 30%, from about 30% to about 50%, from about 30% to about 40%, or from about 40% to about 50% by weight, based on the total weight of the solution. The amount of coating solvent used depends on the coating process and viscosity, as the amount of solvent may affect the uniformity of the drug coating even though the coating solvent will be evaporated.

In other aspects, two or more solvents, two or more therapeutic agents, two or more additives, or, optionally, two or more additional additives may be used in the coating solution or coating mixture. In particular aspects, a polymeric material may be used as an additive in the coating mixture.

Kinase inhibitors and PDE inhibitors may in some instances be lipophilic weak bases insoluble in commonly used organic solvents. In addition, the presence of kinase inhibitors in formulations may change the surface energy of drug/excipient droplets formed by atomization, which bead up but not spread immediately when reaching the device surface. This may lead to non-uniform coatings or uncovered surfaces. In some aspects, utilizing combinations of solvents may mitigate this problem. In some aspects, the mixture of solvents utilized may include two, three, or more solvents. In some aspects, the mixture of solvents utilized may include ethyl acetate, acetone, and DMF.

Various techniques may be used for applying a coating solution or coating mixture to a medical device such as metering, casting, spinning, spraying, dipping (immersing), rolling, ink jet printing, 3D printing, electrostatic techniques, plasma etching, vapor deposition, and combinations of these processes. Choosing an application technique principally depends on the viscosity and surface tension of the coating solution or coating mixture. In some aspects of the present disclosure, metering, dipping and spraying may be preferred because it makes it easier to control the uniformity of the thickness of the drug coating as well as the concentration of the therapeutic agent applied to the medical device. Regardless of whether the coating solution or coating mixture is applied by spraying or by dipping or by another method or combination of methods, additional coating layers may be applied to the medical device in multiple application steps in order to control the uniformity and the amount of therapeutic substance and additive applied to the medical device.

Each applied coating layer may have a thickness from about 0.1 μm to about 15 μm, from about 0.1 μm to about 10 μm, from about 0.1 μm to about 5 μm, from about 0.1 μm to about 1 μm, from about 1 μm to about 15 μm, from about 1 μm to about 10 μm, from about 1 μm to about 5 μm, from about 5 μm to about 15 μm, from about 5 μm to about 10 μm, or from about 10 μm to about 15 μm. The total number of coating layers applied to the medical device is in a range of from about 1 to about 50, from about 1 to about 40, from about 1 to about 30, from about 1 to about 20, from about 1 to about 10, from about 10 to about 50, from about 10 to about 40, from about 10 to about 30, from about 10 to about 20, from about 20 to about 50, from about 20 to about 40, from about 20 to about 30, from about 30 to about 40, or from about 40 to about 50. In some aspects, only one layer is applied to the medical device. In some aspects, more than one layer is applied to the medical device. The total thickness of the coating may be from about 0.1 μm to about 200 μm, from about 0.1 μm to about 150 μm, from about 0.1 μm to about 100 μm, from about 0.1 μm to about 50 μm, from about 0.1 μm to about 10 μm, from about 0.1 μm to about 1 μm, from about 1 μm to about 200 μm, from about 1 μm to about 150 μm, from about 1 μm to about 100 μm, from about 1 μm to about 50 μm, from about 1 μm to about 10 μm, from about 10 μm to about 200 μm, from about 10 μm to about 150 μm, from about 10 μm to about 100 μm, from about 10 μm to about 50 μm, from about 50 μm to about 200 μm, from about 50 μm to about 150 μm, from about 50 μm to about 100 μm, from about 100 μm to about 200 μm, from about 100 μm to about 150 μm, or from about 150 μm to about 200 μm.

In addition to coating layers that comprise the drug coating, the medical device may include one or more intermediate layers or top layers. In some aspects, the intermediate or top layer may be advantageous in order to promote adhesion of the drug coating to the medical device, be an additional layer comprising the additive, or prevent premature drug loss during the device delivery process before deployment at the target site.

As stated previously, in some aspects, the additive may be mixed with the therapeutic agent(s) and/or a coating solvent (or a mixture of coating solvents) to form the coating mixture, which is coated onto the exterior surface of the medical device. Alternatively or additionally, some aspects may include applying the additive to the exterior surface of the medical device separately from the therapeutic agent(s) or the therapeutic agent(s) dissolved in the coating solvent. In some aspects, the additive may be applied to the medical device before the therapeutic agent(s) or the therapeutic agent(s) dissolved in the coating solvent. In some aspects, the additive may be applied to the medical device after the therapeutic agent(s) or the therapeutic agent(s) dissolved in the coating solvent.

In one exemplary aspect, an application device that may be used is a paint jar attached to an air brush, such as a Badger Model 150, supplied with a source of pressurized air through a regulator (Norgren, 0 to 160 psi). When using such an application device, once the brush hose is attached to the source of compressed air downstream of the regulator, the air may be applied. The pressure may be adjusted to approximately 15 psi to 25 psi, and the nozzle condition may be checked by depressing the trigger. Prior to spraying, both ends of a relaxed, expandable medical device may be fastened to the fixture by two resilient retainers, i.e., alligator clips, and the distance between the clips may be adjusted so that the expandable medical device remains in a relaxed condition, for example, a deflated, folded, or an inflated or partially inflated, unfolded condition. The rotor may be then energized and the spin speed adjusted to the desired coating speed, about 40 rpm. With the expandable medical device rotating in a substantially horizontal plane, the spray nozzle may be adjusted so that the distance from the nozzle to the expandable medical device is about 0.2 inch to 4 inches. First, the coating solution or coating mixture may be sprayed substantially horizontally with the brush being directed along the expandable medical device from the distal end of the expandable medical device to the proximal end and then from the proximal end to the distal end in a sweeping motion at a speed such that one spray cycle occurred in about three expandable medical device rotations. The expandable medical device may be repeatedly sprayed with the coating solution, followed by drying, until an effective amount of the drug is deposited on the expandable medical device. It should be understood that this description of an application device, fixture, and spraying technique is exemplary only. Any other suitable spraying or other technique may be used for coating the expandable medical device, particularly for coating the balloon of a balloon catheter or stent delivery system or stent.

In one aspect of the present disclosure, the expandable medical device may be expanded, such as inflated or partially inflated, and the coating solution or coating mixture may be applied to the expanded expandable medical device, for example by spraying, and then the expandable medical device may be dried and subsequently relaxed or collapsed to the unexpanded form or shape. For example, if the expandable medical device is a balloon, the balloon is dried, deflated, and folded. Drying may be performed under vacuum.

After the medical device is sprayed with the coating solution or coating mixture, the coated medical device may be subjected to a drying in which the coating solvent is evaporated. This produces, on the expandable medical device, a coating matrix containing the therapeutic agent and the additive. One example of a drying technique may include placing the coated expandable medical device into an oven at approximately 20 °C or higher for approximately 24 hours or longer, such as up to 48 or 72 hours. Another example may include air drying. Any other suitable method of drying the coating solution may be used. The time and temperature may vary with particular additives and therapeutic agents.

### Medical Device

Aspects of medical devices, including as non-limiting examples balloon catheters and stents will now be described. In the medical devices, a drug coating is applied over an exterior surface of the medical device. Some aspects of methods for preparing the exemplary medical devices will be described subsequently.

### Balloon Catheters

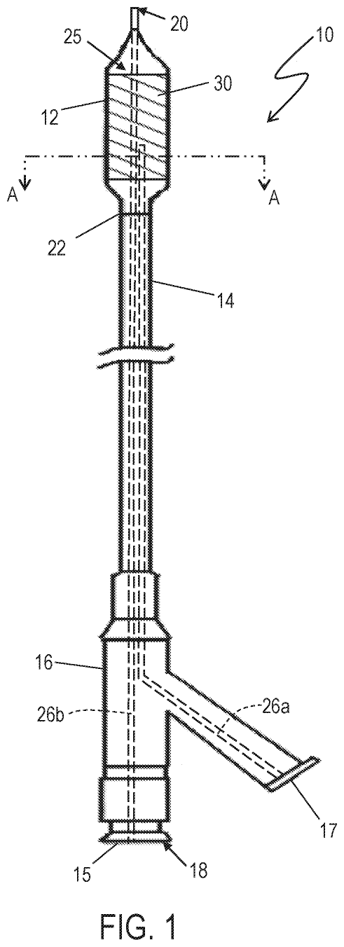

In some aspects, the medical device is a balloon catheter. Referring to the exemplary drawing of FIG. 1, a balloon catheter 10 has a proximal end 18 and a distal end 20. The balloon catheter 10 may be any suitable catheter for desired use, including conventional balloon catheters known to one of ordinary skill in the art. For example, the balloon catheter 10 may be a rapid exchange or over-the-wire catheter. In some specific examples, the balloon catheter may be a ClearStream™ Peripheral catheter available from BD Peripheral Intervention. The balloon catheter 10 may be made of any suitable biocompatible material. The balloon 12 of the balloon catheter may include a polymer material, such as, for example only, polyvinyl chloride (PVC), polyethylene terephthalate (PET), polyethylene, Nylon, PEBAX (i.e. a copolymer of polyether and polyamide), polyurethane, polystyrene (PS), polyethleneterephthalate (PETP), or various other suitable materials as will be apparent to those of ordinary skill in the art.

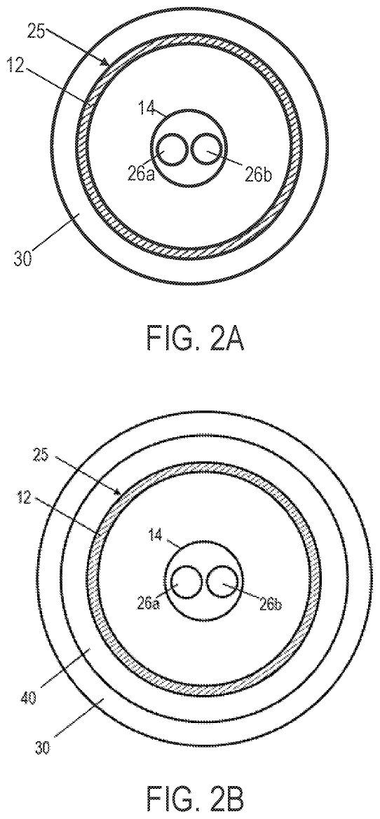

Various aspects of the balloon catheter 10 of FIG. 1 are illustrated through the cross sections along line A-A of FIG. 1 in FIGS. 2A and 2B. Referring jointly to FIGS. 1, 2A, and 2B, the balloon catheter 10 includes an expandable balloon 12 and an elongate member 14. The elongate member 14 extends between the proximal end 18 and the distal end 20 of the balloon catheter 10. The elongate member 14 has at least one lumen 26a, 26b and a distal end 20. The elongate member 14 may be a flexible member which is a tube made of suitable biocompatible material. The elongate member 14 may have one lumen or, as shown in FIGS. 1, 2A, and 2B, more than one lumen 26a, 26b therein. For example, the elongate member 14 may include a guide-wire lumen 26b that extends to the distal end 20 of the balloon catheter 10 from a guide-wire port 15 at the proximal end 18 of the balloon catheter 10. The elongate member 14 may also include an inflation lumen 26a that extends from an inflation port 17 of the balloon catheter 10 to the inside of the expandable balloon 12 to enable inflation of the expandable balloon 12. From the elements of FIGS. 1, 2A, and 2B, even though the inflation lumen 26a and the guide-wire lumen 26b are shown as side-by-side lumens, it should be understood that the one or more lumens present in the elongate member 14 may be configured in any manner suited to the intended purposes of the lumens including, for example, introducing inflation media and/or introducing a guide-wire. Many such configurations are well known in the art.

The expandable balloon 12 is attached to the distal attachment end 22 of the elongate member 14. The expandable balloon 12 has an exterior surface 25 and is inflatable. The expandable balloon 12 is in fluidic communication with a lumen of the elongate member 14, (for example, with the inflation lumen 26a). At least one lumen of the elongate member 14 is configured to receive inflation media and to pass such media to the expandable balloon 12 for its expansion. Examples of inflation media include air, saline, and contrast media.

Still referring to FIG. 1, in one aspect, the balloon catheter 10 includes a handle assembly such as a hub 16. The hub 16 may be attached to the balloon catheter 10 at the proximal end 18 of the balloon catheter 10. The hub 16 may connect to and/or receive one or more suitable medical devices, such as a source of inflation media (e.g., air, saline, or contrast media) or a guide wire. For example, a source of inflation media (not shown) may connect to the inflation port 17 of the hub 16 (for example, through the inflation lumen 26a), and a guide wire (not shown) may be introduced to the guide-wire port 15 of the hub 16, (for example through the guide-wire lumen 26b).

In some examples, the cross section A-A of FIG. 1 may be as depicted according to FIG. 2A, in which the drug coating layer 30 is applied directly onto an exterior surface 25 of the balloon 12. The specific compositions of the drug coating layer 30 itself, according to various aspects, will also be described subsequently in greater detail. In other examples, the cross section A-A of FIG. 1 may be as depicted according to FIG. 2B, in which the drug coating layer 30 is applied onto an intermediate layer 40 overlying the exterior surface 25 of the balloon 12. In some aspects, the exterior surface 25 may undergo a surface modification. In some aspects where the exterior surface 25 is a modified exterior surface, the exterior surface 25 has been subjected to a surface modification, such as a fluorine plasma treatment, which decreases a surface free energy of the exterior surface 25 before application of the drug coating layer 30. Subjecting the exterior surface to a surface modification may decreases the surface free energy of the exterior surface before application of the coating layer and affect the release kinetics of drug in the coating layer from the balloon, the crystallinity of the drug layer, the surface morphology of the coating and particle shape, or the particle size of drug of a therapeutic layer in the coating layer, drug distribution on the surface.

In aspects in which the cross section A-A of FIG. 1 is as depicted according to FIG. 2A, the balloon catheter 10 includes a drug coating layer 30 applied over an exterior surface 25 of the balloon 12. The drug coating layer 30 itself includes a therapeutic agent and an additive. In one particular aspect, the drug coating layer 30 comprises a kinase inhibitor, tyrosine kinase inhibitor, a PDE inhibitor, or an anti-fibrotic therapeutic agent, the polymer, and one or more additional additives. In further aspects, the drug coating layer 30 does not include a polymer.

In other aspects, two or more therapeutic agents are used in combination in the drug coating layer. In other aspects, the device may include a top layer (not shown) overlying the drug coating layer 30. In some aspects, a top coat layer may be advantageous in order to prevent premature drug loss during the device delivery process before deployment at the target site.

### Drug Eluting Stents

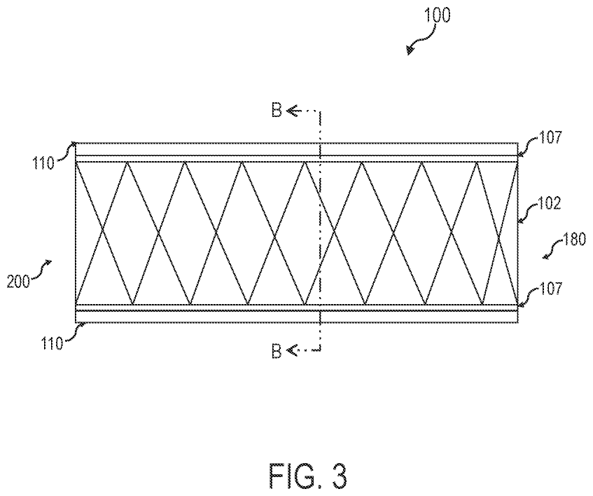

In some aspects, the medical device is drug eluting stent 100. Referring to the example set forth in FIG. 3, a drug eluting stent 100 has a proximal end 180 and a distal end 200. The drug eluting stent 100 may include any suitable base stent 102 for desired use, including conventional stents known to one of ordinary skill in the art. The base stent 102 may be made of any suitable biocompatible metal alloy. Examples of biocompatible metal alloys may include stainless steel, Nitinol or Elgiloy. In some aspects, the shape memory characteristics of Nitinol may allow the base stent 102 to self-expand when placed in a tubular body vessel at normal body temperature.

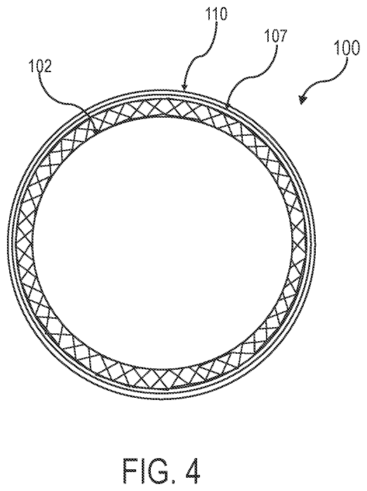

Various aspects of the drug eluting stent 100 of FIG. 3 are illustrated through the cross sections along line B-B of FIG. 3 in FIG. 4. In some examples, the cross section B-B of FIG. 3 may be as depicted according to FIG. 4, in which the drug coating layer 110 is applied directly onto an exterior surface 107 of the base stent 102. In some aspects subsequently described, the exterior surface 107 may undergo a surface modification. In aspects where the exterior surface 107 is a modified exterior surface, the exterior surface 107 has been subjected to a surface modification, such as a fluorine plasma treatment, which decreases a surface free energy of the exterior surface 107 before application of the drug coating layer 110. Subjecting the exterior surface to a surface modification may decreases the surface free energy of the exterior surface before application of the coating layer and affect the release kinetics of drug in the coating layer from the balloon, the crystallinity of the drug layer, the surface morphology of the coating and particle shape, or the particle size of drug of a therapeutic layer in the coating layer, drug distribution on the surface.

In aspects in which the cross section B-B of FIG. 3 is as depicted according to FIG. 4, the drug eluting stent 100 includes a drug coating layer 100 applied over an exterior surface 107 of the base stent 102. The drug coating layer 110 itself includes a therapeutic agent and an additive. In one particular aspect, the drug coating layer 110 comprises a kinase inhibitor or a tyrosine kinase inhibitor, a PDE inhibitor, an anti-fibrotic drug therapeutic agent, a polymer, and one or more additional additives. In further aspects, the drug coating layer 110 does not include a polymer.

In other aspects, two or more therapeutic agents are used in combination in the drug coating layer 110. In other aspects, the device may include a top layer (not shown) overlying the drug coating layer 100. In some aspects, a top coat layer may be advantageous in order to prevent premature drug loss during the device delivery process before deployment at the target site.

### EXAMPLES

### Example 1

In Example 1, two formulations were prepared as subsequently described and summarized in Table 1.

To prepare Formulation 1, 50 mg of sunitinib malate were weighed and dissolved in 5.14 mL of N,N-Dimethylformamide (DMF) (HPLC grade) in an amber vial, which was then bath sonicated for 5 min to form a clear yellow solution. Separately, 300 mg of PVDF-HFP were added to 21.6 mL of acetone containing 1.8 mg BHT. This mixture was bath sonicated for 15 min to entirely dissolve the solid PVDF-HFP. Next, 18 mL of the PVDF-HFP/acetone solution were transferred and mixed with the prepared sunitinib/DMF solution, followed by the addition of 36 mL of methyl acetate. This formulation was stored at 4 °C before spray coating.

To prepare Formulation 2, 70 mg of sunitinib malate were weighed and dissolved in 5.6 mL of DMF (HPLC grade) in an amber vial, which was then bath sonicated for 5 min to form a clear yellow solution. Separately, 300 mg of PVDF-HFP were added in 24 mL of acetone containing 3 mg of BHT, which was bath sonicated for 15 min to entirely dissolve the solid PVDF-HFP. Next, 16.8 mL of the PVDF-HFP/acetone solution was transferred and mixed with the prepared sunitinib/DMF solution, followed by the addition of 33.6 mL of methyl acetate. This formulation was stored at 4 °C before spray coating.

TABLE 1
Formulation 1 and Formulation 2.

| | Formulation 1 | Formulation 2 |
|---|---|---|
| Polymer | PVD-HFP | PVD-HFP |
| Drug | Sunitinib malate | Sunitinib malate |
| Polymer-to-drug | 5:1 | 3:1 |
| ratio (wt/wt) | | |
| Solvents | Methyl acetate/ | Methyl acetate/ |
| (v/v/v) | Acetone/DMF | Acetone/DMF |
| | (6:3:1) | (6:3:1) |
| BHT | 3 wt. % of loaded drug | 3 wt. % of loaded drug |
| Concentration | 5 mg/mL | 5 mg/mL |
| of total solid | | |

The Formulations 1 and 2 were then coated on life stents using a Sono-Tek Extracoat ultrasonic spray coating system with the parameters summarized in Table 2. Formulations 1 and 2 were used to produce Sample Stent 1 and Sample Stent 2, respectively. The primer was 5 mg/mL PBMA in mixed solvents (Acetone/Cyclohexanone=9:1)

TABLE 2
Coating Parameters of Sample Stent 1 and Sample Stent 2.

| | Sample Stent 1 | | Sample Stent 2 | |
|---|---|---|---|---|
| Primer | PBMA | | PBMA | |
| Flow Rate | 0.09 | mL/min | 0.09 | mL/min |
| Power | 0.8 | W | 0.8 | W |
| Shaping Air | 5 | L/min | 5 | L/min |
| Rotational Speed | 100 | rpm | 100 | rpm |
| Translation Speed | 5 | mm/s | 5 | mm/s |
| Nozzle-to-stent distance | 15 | mm | 15 | mm |

| Spray loops | 14 | 14 |
|---|---|---|
| Formulation | Formulation 1 | Formulation 2 |

| | | | | |
|---|---|---|---|---|
| Flow Rate | 0.09 | mL/min | 0.09 | mL/min |
| Power | 0.8 | W | 0.8 | W |
| Shaping Air | 5 | L/min | 5 | L/min |
| Rotational Speed | 100 | rpm | 100 | rpm |
| Translation Speed | 5 | mm/s | 5 | mm/s |
| Nozzle-to-stent distance | 15 | mm | 15 | mm |

| | | |
|---|---|---|
| Spray loops | 159 | 153 |

| | | | | |
|---|---|---|---|---|
| Targeted drug dose | 0.5 | mg | 0.75 | mg |

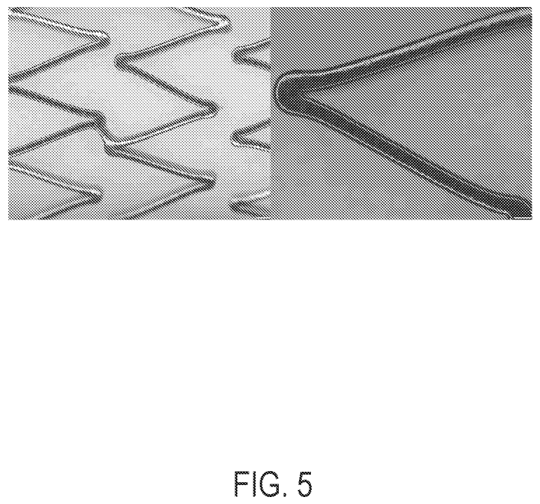

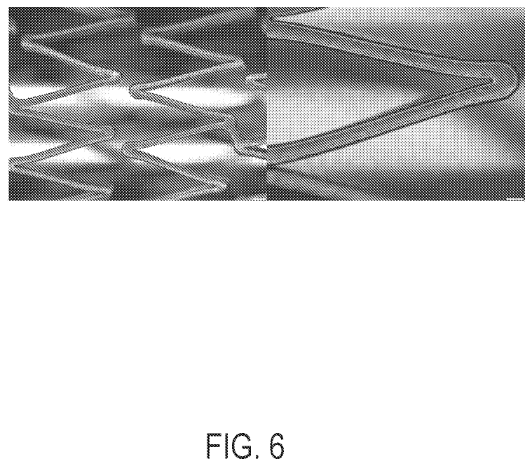

As shown in FIGS. 5 and 6, Sample Stent 1 and Sample Stent 2 had smooth, uniform coatings formed on the struts without noticeable coating defects.

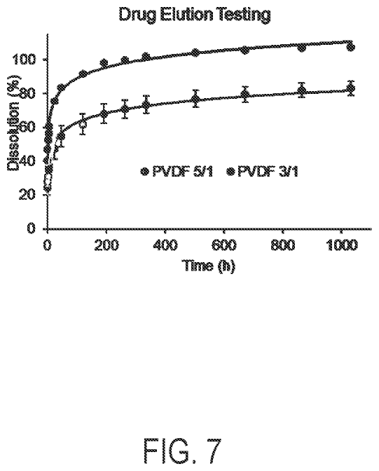

To evaluate the in vitro drug release profiles of Sample Stent 1 and Sample Stent 2, drug elution testing was performed in 1 time phosphate buffered saline (with a pH between 7.3 and 7.4) as the elution media at 37 °C. The results are shown in FIG. 7

As shown in FIG. 7, a burst release was found for both Formulation 1 and Formulation 2, followed by sustained release kinetics over greater than 45 days. A slower release rate was observed in Formulation 1, where approximately 20% of unreleased sunitinib resided on the stent after elution for 45 days.

### Example 2

Stents of sizes 5×40 and 6×40 were prepared either with the Formulation 1 or 2 as set forth above and inserted in the peripheral arteries of healthy Yorkshire swine and examined at varying time points.

At 28 days post stent insertion the effects on the cross-sectional vessel area were evaluated and compared with several other models. Comparative A was a bare metal stent, Comparative B was the Orsiro stent (sirolimus eluting stent) commercially-available from Biotronik, and Comparative C was the Eluvia stent (paclitaxel eluting stent) commercially-available from Boston Scientific.

The results of the study are presented subsequently in Tables 3 and 4.

TABLE 3
Morphometric comparison of cross-sectional vessel areas and
neointimal responses of coronary arteries treated with Sample
Stents 1 and 2 and Comparative Stents A, B, and C.

| | | EEL | IEL | Lumen | Med | Neoint | % |
|---|---|---|---|---|---|---|---|
| | | Area | Area | Area | Area | Area | Stenosis |
| | N | (mm²) | (mm²) | (mm²) | (mm²) | (mm²) | (%) |
| Sample 1 | 4 | 24.61 | 22.06 | 18.23 | 2.55 | 3.83 | 18.16 |
| Sample 2 | 4 | 27.05 | 24.28 | 18.95 | 2.78 | 5.33 | 21.62 |
| Compar- | 6 | 33.53 | 29.63 | 24.36 | 3.91 | 5.26 | 18.09 |
| ative A | | | | | | | |
| Compar- | 4 | 11.27 | 9.75 | 7.45 | 1.73 | 2.09 | 21.48 |
| ative B | | | | | | | |
| Compar- | 4 | 35.36 | 32.46 | 23.93 | 2.90 | 8.53 | 25.99 |
| ative C | | | | | | | |
| P value | | <0.001 | <0.001 | <0.001 | <0.001 | 0.001 | 0.41 |

EEL = external elastic lamina, IEL = internal elastic lamina, Lumen Area = cross sectional area of vessel lumen, Med Area = EEL Area − IEL Area, Neoint Area = IEL Area − Lumen Area (representing cross sectional area consisting of neointimal tissue), % Stenosis = [1 − (Lumen Area/IEL Area)] * 100

TABLE 4
Semi-quantitate scoring for arterial injury, fibrin, malapposition,
hemorrhage, and endothelial cell loss for histologic
sections of coronary arties treated with Sample Stents
1 and 2 and Comparative Stents A, B, and C.

| | Sam- | Sam- | Comp. | Comp. | Comp. | P |
|---|---|---|---|---|---|---|
| | ple 1 | ple 2 | A | B | C | value |
| N | 4 | 4 | 6 | 4 | 4 | |
| Injury Score | 0.91 | 0.78 | 0.76 | 0.7 | 0.98 | 0.026 |
| Struts with Fibrin | 47.14 | 64.97 | 34.3 | 94.77 | 98.44 | 0.002 |
| (%) | | | | | | |
| Fibrin Score | 1.31 | 1.56 | 1.17 | 2.5 | 3 | 0.006 |
| Malapposition (%) | 5.09 | 5.22 | 7.18 | 13.47 | 64.46 | 0.045 |
| Struts with RBCs | 0 | 10.52 | 1.66 | 6.67 | 0 | 0.025 |
| Endothel Loss Score | 1.25 | 0.44 | 1.5 | 0.25 | 1.81 | 0.14 |
| Struts with | 0 | 0 | 0 | 0 | 0 | 1 |
| Granulomas (%) | | | | | | |
| Uncovered Struts | 27.04 | 10.47 | 42.2 | 8.4 | 6.67 | 0.47 |
| (%) | | | | | | |
| I/M Inflammation | 0 | 0.13 | 0.04 | 0 | 0.44 | 0.06 |
| Score | | | | | | |
| Adventitial | 0 | 0 | 0 | 0 | 0 | 1 |
| Inflammation Score | | | | | | |
| Struts with Giant | 0 | 3.87 | 0.25 | 0 | 0.75 | 0.21 |
| Cells (%) | | | | | | |
| Calcification (%) | 0 | 0 | 0 | 0 | 0 | 1 |

The P values shown are calculated based on all the data for the four groups in the table. A number below 0.05 indicates significant differences between the means for the four groups.

As shown in Tables 3 and 4, the overall mean percentage of stenosis was minimal for Samples 1 and 2 (e.g., for Sample 2, stenosis %=21.62±6.68), which was comparable to the results obtained with other the Comparative Stents A, B, and C tested in the same animal model. For Sample 2, mild to moderate fibrin was noted in 64.97-20.84% of struts with mean fibrin scores of 1.56-0.83, intimal/medial inflammation was essentially absent. There was no incidence of granulomas and giant cells were minimal. Also for Sample 2, hemorrhage around struts was unremarkable and there was absence of calcification, with no incidence of adventitial inflammation. The percentage of uncovered struts was 10.47±12.13%. There was minimal endothelial loss (mean endothelial loss scores=0.44±0.52%). To the contrary, endothelial coverage was poor with mean endothelial loss scores of 1.81±0.24% in the vessels treated with Comparative Sample C, which comprised paclitaxel.

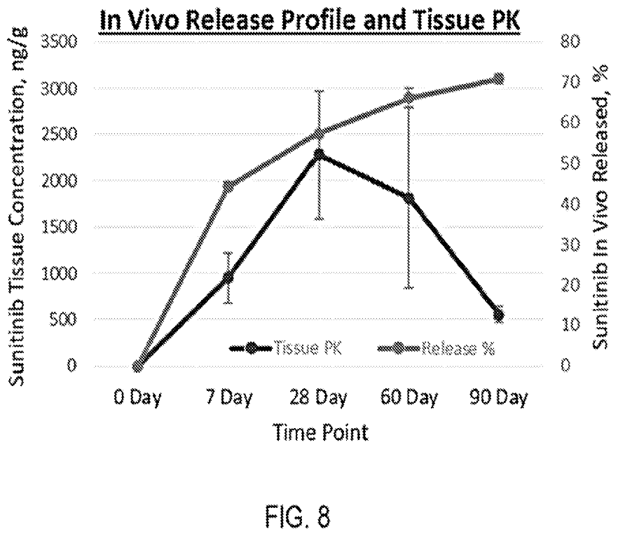

The studies were then extended out to 60 and 90 days, followed by histological and pharmacokinetic profile analysis. Formulation 1 was prepared with a dose density of 1 μg/mm and a total dose of 425 μg/stent and Formulation 2 was at a dose density of 2 μg/mm and a total dose of 750 μg/stent. FIG. 8 shows both the release profile and pharmacokinetic (PK) profiles at the 7, 28, 60 and 90 day time points after stent insertion. Further, as with above, the morphometric cross-section vessel areas were measured at the 60 and 90 day time points and compared with Comparative A and C as set forth above. Tables 5 and 6 shows the results at 60 days and Tables 7 and 8 show the results at 90 days.

TABLE 5
Morphometric comparison of cross-sectional vessel areas and
neointimal responses of coronary arteries treated with Sample
Stents 1 and 2 and Comparative Stents A and C for 60 days.

| | | EEL | IEL | Lumen | Med | Neoint | % |
|---|---|---|---|---|---|---|---|
| | | Area | Area | Area | Area | Area | Stenosis |
| | N | (mm²) | (mm²) | (mm²) | (mm²) | (mm²) | (%) |
| Sample 1 | 4 | 24.43 | 20.93 | 10.41 | 3.51 | 10.51 | 46.51 |
| Sample 2 | 4 | 21.54 | 19.23 | 14.90 | 2.31 | 4.32 | 22.86 |
| Compar- | 3 | 28.41 | 26.09 | 12.13 | 2.32 | 13.96 | 51.96 |
| ative A | | | | | | | |
| Compar- | 4 | 36.14 | 32.39 | 23.26 | 3.75 | 9.13 | 27.83 |
| ative C | | | | | | | |
| P value | | 0.0003 | <0.0001 | 0.0203 | 0.2168 | 0.3422 | 0.3147 |

The P values shown are calculated based on all the data for the four groups in the table. A number below 0.05 indicates significant differences between the means for the four groups.

TABLE 6
Semi-quantitate scoring for arterial injury, fibrin, malapposition,
hemorrhage, and endothelial cell loss for histologic sections
of coronary arteries treated with Sample Stents 1 and 2
and Comparative Stents A and C at 60 days.

| | Sample | Sample | Comp. | Comp. | P |
|---|---|---|---|---|---|
| | 1 | 2 | A | C | value |
| N | 4 | 4 | 3 | 4 | |
| Injury Score | 0.92 | 0.94 | 1.17 | 0.78 | 0.5361 |
| Struts with Fibrin (%) | 48.55 | 24.37 | 1.35 | 86.98 | 0.0206 |
| Fibrin Score | 1.50 | 0.56 | 0.00 | 2.81 | 0.0208 |
| Malapposition (%) | 2.08 | 0.00 | 0.00 | 57.68 | 0.0073 |
| Struts with RBCs | 20.05 | 0.2 | 2.87 | 2.63 | 0.5526 |
| Endothelialization | 3.00 | 3.00 | 3.00 | 2.94 | 0.4318 |
| Score | | | | | |
| Struts with | 0.00 | 0.00 | 22.64 | 0.00 | 0.2615 |
| Granulomas (%) | | | | | |
| Uncovered Struts (%) | 0.00 | 0.72 | 0.00 | 16.22 | 0.0536 |
| I/M Inflammation | 0.75 | 0.25 | 1.75 | 0.88 | 0.2412 |
| Score | | | | | |
| Adventitial | 0.38 | 0.00 | 0.00 | 0.00 | 0.1169 |
| Inflammation Score | | | | | |
| Struts with Giant Cells | 5.56 | 12.01 | 11.94 | 2.92 | 0.5406 |
| (%) | | | | | |
| Calcification (%) | 0.6 | 0.00 | 0.00 | 0.00 | 0.4318 |

TABLE 7
Morphometric comparison of cross-sectional vessel areas and
neointimal responses of coronary arteries treated with Sample
Stents 1 and 2 and Comparative Stents A and C for 90 days.

| | | EEL | IEL | Lumen | Med | Neoint | % |
|---|---|---|---|---|---|---|---|
| | | Area | Area | Area | Area | Area | Stenosis |
| | N | (mm²) | (mm²) | (mm²) | (mm²) | (mm²) | (%) |
| Sample 1 | 4 | 23.26 | 21.34 | 15.15 | 1.92 | 6.19 | 26.93 |
| Sample 2 | 4 | 23.93 | 22.3 | 18.35 | 1.63 | 3.95 | 17.67 |
| Compar- | 4 | 32.15 | 29.13 | 14.8 | 3.02 | 14.33 | 49.92 |
| ative A | | | | | | | |
| Compar- | 4 | 37.6 | 33.78 | 18.77 | 3.82 | 15.01 | 45.36 |
| ative C | | | | | | | |
| P value | | 0.0006 | 0.0008 | 0.2233 | 0.0015 | 0.0001 | 0.0014 |

TABLE 8
Semi-quantitate scoring for arterial injury, fibrin, malapposition,
hemorrhage, and endothelial cell loss for histologic sections
of coronary arties treated with Sample Stents 1 and 2 and
Comparative Stents A and C at 90 days.

| | Sample | Sample | Comp. | Comp. | P |
|---|---|---|---|---|---|
| | 1 | 2 | A | C | value |
| N | 4 | 4 | 4 | 4 | |
| Injury Score | 1.00 | 0.99 | 0.99 | 0.96 | 0.5153 |
| Struts with Fibrin (%) | 2.8 | 0.72 | 22.91 | 70.62 | 0.0126 |
| Fibrin Score | 0.06 | 0.00 | 0.75 | 2.25 | 0.0537 |
| Malapposition (%) | 0.00 | 0.00 | 25.00 | 58.64 | 0.0204 |
| Struts with RBCs | 0.00 | 0.00 | 1.59 | 1.67 | 0.3109 |
| Endothelialization | 3.00 | 3.00 | 2.88 | 2.5 | 0.0461 |
| Score | | | | | |
| Struts with | 0.00 | 0.00 | 0.00 | 2.56 | 0.4262 |
| Granulomas (%) | | | | | |
| Uncovered Struts (%) | 0.00 | 0.00 | 1.49 | 10.96 | 0.1114 |
| I/M Inflammation | 0.56 | 0.06 | 1.63 | 1.81 | 0.0255 |
| Score | | | | | |
| Adventitial | 0.00 | 0.00 | 0.00 | 0.00 | 1.000 |
| Inflammation Score | | | | | |
| Struts with Giant Cells | 20.13 | 12.47 | 10.62 | 6.48 | 0.1923 |
| (%) | | | | | |
| Calcification (%) | 0.2 | 0.62 | 0.00 | 0.00 | 0.2333 |

The P values shown are calculated based on all the data for the four groups in the table. A number below 0.05 indicates significant differences between the means for the four groups.

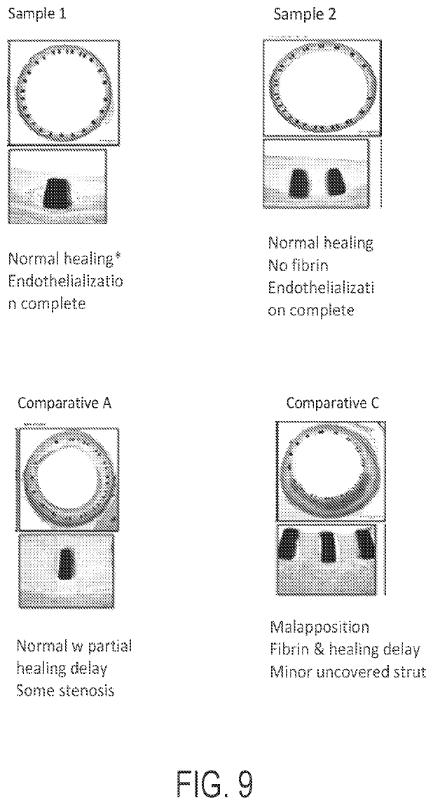

As shown in Tables 5-8, the overall mean percentage of restenosis for Samples 1 and 2 continued to be minimal at 60 and 90 days, while for Comparatives A and C, stenosis increased notably. The fibrin decreased from the earlier time points in Sample 2, as did the fibrin scores, while intimal/medial inflammation only slightly increased. There was no incidence of granulomas and no identification of malapposition. Both the bare stent (Comparative A) and the paclitaxel stent (Comparative B) showed significant malposition. Similarly, both Sample 1 and 2 showed no uncovered struts, while the paclitaxel stent showed an incidence of 16.22%. FIG. 9 shows cross-section views of Comparative A and C with Samples 1 and 2, showing the paclitaxel stent to have malapposition, with fibrin and healing delay. In contrast, both Sample 1 and Sample 2 show normal healing, with no malapposition, no fibrin and complete endothelialization.

### Example 3

Drug elution from balloons was also examined to determine profiles for release. PLGA was selected as a biopolymer with sunitinib as the tyrosine kinase inhibitor. The tyrosine kinase inhibitor was examined in both microparticle form, in crystalline form and in the presence of one or more excipients. The tyrosine kinase was also examined as a free base and as ionized forms with malate and hemi-pamoate. The combination of PLGA with a sunitinib malate and sodium docusate as excipient was studied for how different PLGA compositions might affect the elution profile.

To prepare the drug coating, microparticles with sunitinib malate and PLGA (PLGA/sunitinib microparticles) were prepared by emulsion evaporation/extraction with a homogenizer in polyvinyl alcohol, followed by centrifugation and vacuum drying.

Table 10 shows an elution profile of sunitinib from the identified drug coating compositions.

TABLE 10
Loading properties for varied PLGA/sunitinib/sodium docusate compositions.

| | Loading | PLGA | PLGA:sunitinib:sodium | DMSO:DCM | |
|---|---|---|---|---|---|
| Sample | Dose % | Used | docusate ratio | ration | Vo:Vw |
| 1 | 16.69 | 753H | 3:1:1 | 1:2 | 2.25:10 |
| 2 | 16.26 | 753H | 3:1:1 | 1:6 | 3.5:10 |
| 3 | 17 | 753H | 3:1:1 | 1:4 | 3.75:10 |
| 4 | 16.44 | 753H | 3:1:1 | 1:8 | 6.75:10 |
| 5 | 21.3 | 753H | 2:1:1 | 1:4 | 3.75:10 |
| 6 | 15.78 | 753H | 3:1:1 | 1:4 | 3.75:10 |
| 7 | 16.51 | 757S | 3:1:1 | 1:4 | 3.75:10 |
| 8 | 15.38 | 753S | 3:1:1 | 1:4 | 3.75:10 |
| 9 | 24.61 | 753S | 3:2:2 | 1:4 | 3.75:10 |
| 10 | 24.59 | 756S | 3:2:2 | 1:4 | 3.75:10 |
| 11 | 26.55 | 753S | 3:2:2 | 1:4 | 3.75:40 |
| 12 | 21.5 | 753H | 2:1:1 | 1:4 | 3.75:50 |

As shown in Table 10, the ability to load more of the tyrosine kinase inhibitor in the PLGA microparticles was increased when the presence of sodium docusate was higher. These suggest that excipients can assist in increasing drug loading for the drug coating.

Drug coatings with crystalline forms of tyrosine kinase inhibitors were also examined. Sunitinib was again chosen as the candidate drug and crystal pamoate salts were prepared by recrystaliization in ethanol or ethanol and DMF for a hemi-pamoate. Sunitinib as a free base has a solubility in PBS of 26.4 μmol/L, whereas the malate salt has a solubility of 1231 μmol/L. The pamoate salts further lowered the solubility of the free base, with the pamoate crystal having a solubility of 9.6 μmol/L and the hemi-pamoate having a solubility of 6.02 μmol/L.

### Example 4

In Example 4, formulations for stent drug coatings were prepared as subsequently described, followed by obtaining 7, 28, 60, and 90 days histological data from in vivo studies when implanted in femoral arteries of porcine model.

Five total formulations were used: a control of PVDF only, prepared at a concentration of 5 mg/mL in a solution of 90% acetone and 10% cyclohexane; a colchicine coating, prepared in a 5:1 PVDF: colchicine ratio with 5 mg/mL provided to 90% acetone and 10% DMF; a roflumilast coating at a 5:1 PVDF:roflumilast ratio, with 7.5 mg?1 provided to a 42.5% EtOAc, 50% acetone, and 7.5% DMF solution; a tadalafil coating prepared at 3:1 PVDF:tadalafil, provided at 5 mg/mL in a solution of 30% EtOAc, 60% acetone, and 10% DMF; and a sunitinib coating provided at 3:1 PVDF:sunitinib malate, prepared at 5 mg/mL in a solution of 60% MeOAc, 30% acetone, and 10% DMF. Coatings were provided to 5×40 mm and 6×40 mm stents. Loading concentrations were assessed prior to and after ETO (ethylene oxide) sterilization and no discernable change was noted by the process.

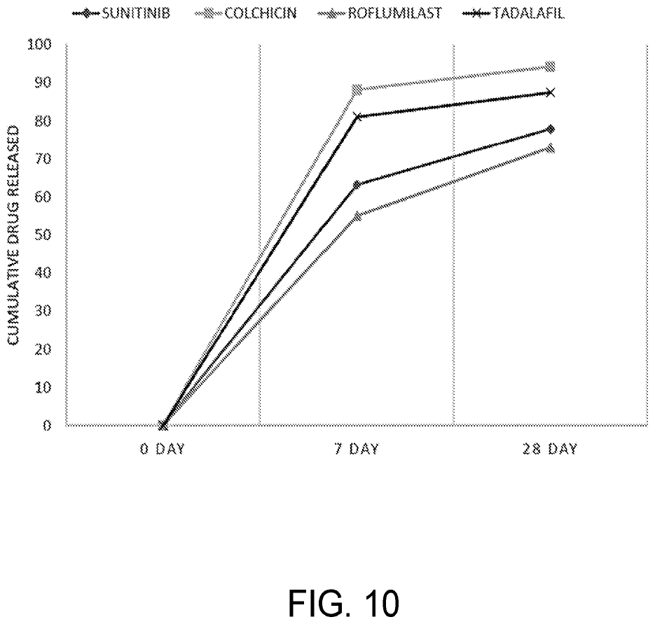

FIG. 10 sets forth some preliminary pharmacokinetic data obtained from the prepared stents at 7 and 28 days after implantation within the pigs. Colchicine and roflumilast demonstrated expected target ranges, with tadalafil providing lower than expected based on comparison to oral bioavailability.

Histological studies were then performed at 60 and 90 days. Table 11 provides some observed data from each of the five studied groups.

TABLE 11
(areas in mm²)

| | | | | | Neoin- | % |
|---|---|---|---|---|---|---|
| | EEL | IEL | Lumen | Medial | timal | Ste- |
| Treatment | area | area | Area | Area | Area | nosis |
| Colchicine | 22.27 ± | 20.71 ± | 6.77 ± | 1.56 ± | 13.94 ± | 67.45 ± |
| | 4.08 | 3.7 | 1.63 | 0.46 | 2.29 | 3.94 |
| Roflumilast | 25.39 ± | 23.13 ± | 13.19 ± | 2.27 ± | 9.94 ± | 40.45 ± |
| | 4.31 | 3.79 | 2.45 | 0.57 | 6.18 | 20.06 |
| Sunitinib | 25.47 ± | 22.91 ± | 14.86 ± | 2.56 ± | 8.05 ± | 33.76 ± |
| | 4.8 | 3.98 | 4.18 | 0.85 | 5.83 | 20.3 |
| Tadalafil | 25.28 ± | 22.95 ± | 14.98 ± | 2.33 ± | 7.97 ± | 35.38 ± |
| | 3.74 | 3.39 | 6.72 | 0.95 | 5.23 | 26.08 |
| PVDF | 25.67 ± | 23.62 ± | 12.41 ± | 2.06 ± | 11.2 ± | 43.59 ± |
| only | 4.7 | 3.95 | 5.77 | 0.77 | 9.35 | 31.73 |

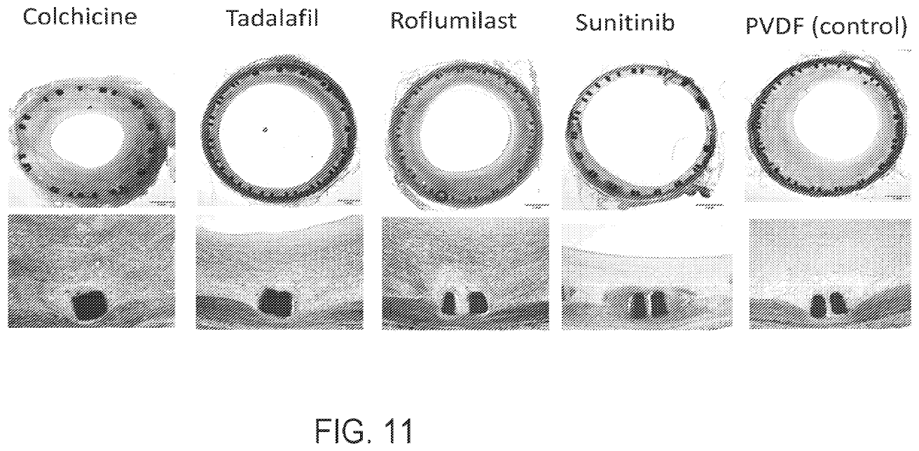

From these data, the percentage of stenosis between sunitinib and tadalafil were similar, with colchicine demonstrating a seemingly accelerated amount of stenosis. FIG. 11 shows overall cross-sectional views and magnified views of the amount of inflammation seen. Table 12 provides a further summary of pertinent histological data.

TABLE 12

| | Colchi- | Tadal- | | | PVDF |
|---|---|---|---|---|---|
| | cine | afil | Roflumilast | Sunitinib | only |
| Restenosis % | 67.45 | 35.38 | 40.45 | 33.76 | 43.59 |
| area | | | | | |
| PGs/Collagen | 3.13 | 1.44 | 0.75 | 1.06 | 1.13 |
| score | | | | | |
| Fibrin % | 72.2 | 26.69 | 16.65 | 33.17 | 16.96 |
| Inflammation | 3.06 | 1.19 | 0.69 | 1.19 | 1.19 |
| Score | | | | | |
| Giant cell % | 18.76 | 7.26 | 0.49 | 4.14 | 4.36 |

These data were also repeated at 90-days. Table 13 sets forth the same collected as Table 11 and Table 14 sets forth the same collected data as in Table 12.

TABLE 13

| | | | | | Neoin- | % |
|---|---|---|---|---|---|---|
| | EEL | IEL | Lumen | Medial | timal | Ste- |
| Treatment | area | area | Area | Area | Area | nosis |
| Colchicine | 21.45 ± | 20.23 ± | 3.71 ± | 1.22 ± | 16.52 ± | 81.83 ± |
| | 4.33 | 4.08 | 3.02 | 0.26 | 4.28 | 15.77 |
| Roflumilast | 24.31 ± | 22.2 ± | 15.44 ± | 2.1 ± | 6.77 ± | 30.69 ± |
| | 4.46 | 4.36 | 4.69 | 0.26 | 2.67 | 11.16 |
| Sunitinib | 21.25 ± | 19.31 ± | 13.28 ± | 1.94 ± | 6.03 ± | 30.77 ± |
| | 3.9 | 3.41 | 1.9 | 0.52 | 2.13 | 6.89 |
| Tadalafil | 22.81 ± | 21 ± | 15.09 ± | 1.81 ± | 5.91 ± | 27.4 ± |
| | 3.6 | 3.03 | 1.96 | 0.68 | 3.35 | 11.56 |
| PVDF | 26.03 ± | 24.05 ± | 15.35 ± | 1.98 ± | 8.7 ± | 34.97 ± |
| only | 4.39 | 3.91 | 5.63 | 0.65 | 6.25 | 23.26 |

TABLE 14

| | Colchi- | Tadal- | | | PVDF |
|---|---|---|---|---|---|
| | cine | afil | Roflumilast | Sunitinib | only |
| Restenosis % | 81.83 | 27.4 | 30.69 | 30.77 | 34.97 |
| area | | | | | |
| PGs/Collagen | 3.38 | 1.38 | 1.44 | 1.31 | 1.25 |
| score | | | | | |
| Fibrin % | 3.13 | 1.31 | 1.0 | 1.0 | 1.0 |
| Inflammation | 1.44 | 1.94 | 0.56 | 0.25 | 1.25 |
| Score | | | | | |
| Giant cell % | 11.7 | 30.12 | 0.0 | 2.03 | 2.35 |

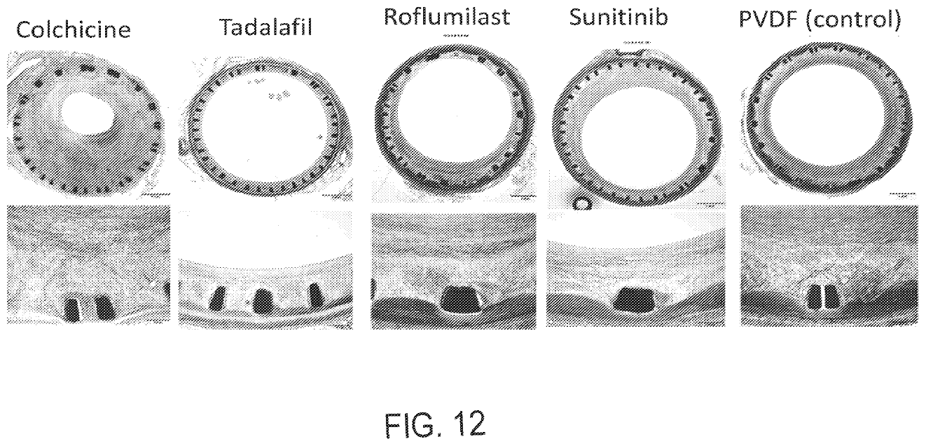

These data reflect that tadalafil and sunitinib both offer improvements in the amount of restenosis observed. Similar cross sectional images are set forth in FIG. 12.

### Example 5

In Example 5, formulations with tadalafil and sildenafil were prepared as balloon coatings as subsequently described.

To prepare PLGA/tadalafil microparticles, and oil/water emulsion and evaporation method was used. PLGA 755S was mixed with tadalafil in DMSO in the desired ratio. For example, for a ratio of 4:1, 60 mg of tadalafil and 240 mg of PLGA 755S were added into 1 mL of DMSO and 8 mL of dichloromethane (DCM). The organic solution is added to an aqueous solution of 5% polyvinyl alcohol (PVA) in water that has been pre-saturated by DCM. The mixture was then emulsified (VWR 250 homogenizer with a VWR Saw-Tooth Generator Probe 20×11 mm) for 1 min to form the emulsion. The emulsion was added to 250 mL of 2% PVA and allowed to stir continuously overnight (at 500 rpm) to allow the organic solvent to evaporate. Resulting suspensions were centrifuged (at 4000 g) and washed three times with deionized water. Microparticles were dried in a vacuum oven at room temperature.

For sildenafil microparticles, a similar method was utilized with the additional step of encapsulating the more water soluble sildenafil prior to adding into organic solvents. 120 mg of sildenafil citrate and 120 mg of sodium docusate were dissolved in 1.5 mL while 240 mg of PLGA 753H in 6 mL was prepared and then the two were combined. The resulting solution was vortexed for 1 mL and then poured in 300 mL 1% PVA in water and then left for the solvent to evaporate overnight. Resulting microparticles were washed with water, centrifuged (4000 g for 8 min) and dried in vacuum over.

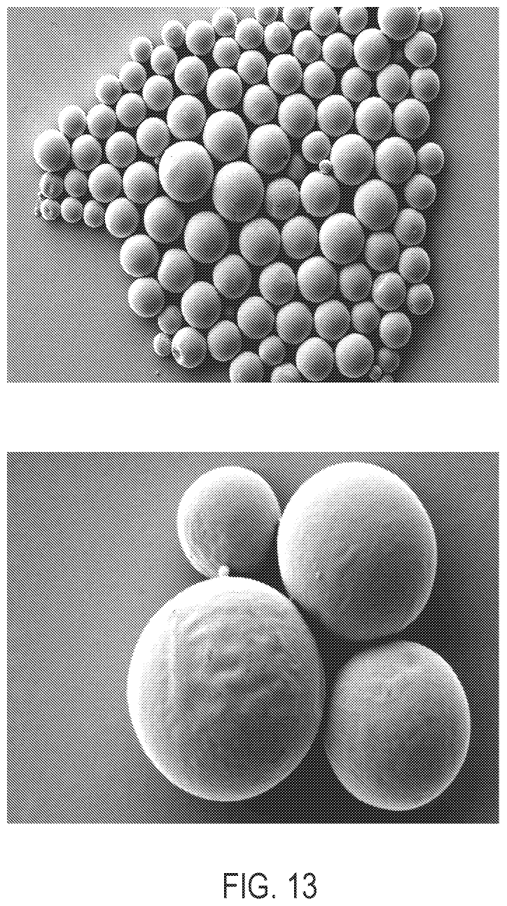

For comparison, microparticles with roflumilast, sunitinib and colchicine were are also prepared in a similar manner. Representative scanning electron microscopy images of the microparticles prepared with sunitinib and PLGA are seen in FIG. 13. These were prepared using the described oil in water emulsion evaporation method. As is seen, the microparticles show a spherical morphology (FIG. 13 Top Panel) at low magnification and (FIG. 13 Bottom Panel) at high magnification.

While particular aspects have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

1-30. (canceled)

31. A stent, stent graft or other permanent or semi-permanent medical device for delivering a therapeutic agent to a blood vessel, comprising a device body and a drug coating thereon, wherein the drug coating comprises:
a therapeutic agent and at least one of a biodurable polymer, and an excipient, wherein:
the therapeutic agent comprises a kinase inhibitor, an anti-fibrotic drug, or mixtures thereof,
the biodurable polymer is chosen from poly(vinylidene hexafluoropropylene) (PVDF-HFP), polyethylene terephthalate (PET), nylon 6,6, polyurethane (PU), polytetrafluoroethylene (PTFE), polyethylene, polysiloxanes (silicones) and poly(methylmethacrylate) (PMMA), and combinations thereof; and
an excipient chosen from a fatty acid, a fatty acid ester, polylactic acid (PLLA,PDLA,PDLLA), polycaprolactone (PCL), sodium docusate, PLGA, PLGA-b-mPEG, polyglutamic acid, polyacrilic acid, hyaluronic acid, alginate, PVA, PVP, Pluronic (PEO—PPO-PEO), cellulose, CMC, HPC, starch, chitosan, human serum albumin (HSA), phospholipids, fatty acid, fatty acid esters, triglycerides, beeswax, cyclodextrin, Tween 20, Tween 80, TPGS, SLS, butylated hydroxytoluene, vitamin E, vitamin E succinate, and sorbitol esters.

32. The stent, stent graft or other permanent or semi-permanent medical device of claim 31, wherein the kinase inhibitor is chosen from bosutinib, ceritinib, crizotinib, gefitinib, ruxolitinib, imatinib, axitinib, nilotinib, trametinib, afatinib, ibrutinib, cabozantinib, imatinib, lenvatinib, sunitinib, regorafenib, sorafenib, vandetanib, dasatinib, pazopanib, triamciclone, tranilast, halofuginone, montelukast, zafirlukast, pirfenidone, Y27632, CA3, verteporfin, VGLL4 peptide, nintedanib, avapritinib, abemaciclib, erdafitinib, fedratinib, palbociclib, and pemigatinib.

33. The stent, stent graft or other permanent or semi-permanent medical device of claim 31, wherein the kinase inhibitor is sunitinib.

34. The stent, stent graft or other permanent or semi-permanent medical device of claim 31, wherein the kinase inhibitor is in the form of a free base, a crystal, a free acid or a salt.

35. The stent, stent graft or other permanent or semi-permanent medical device of claim 34, wherein the salt is of a hydrochloride salt, a sodium salt, a sulfate salt, an acetate salt, a phosphate and/or diphosphate sale, a pamoate or hemi-pamoate salt, a chloride salt, a potassium salt, a maleate salt, a calcium salt, a citrate salt, a mesylate salt, a nitrate salt, a tartrate salt, an aluminum salt, a stearic acid salt, a dioctyl sulfosuccinic acid or a gluconate salt.

36. The stent, stent graft or other permanent or semi-permanent medical device of claim 31, wherein the anti-fibrotic drug is chosen from triamciclone, tranilast, halofuginone, montelukast, zafirlukast, pirfenidone, nintedanib, and combinations thereof.

37. The stent, stent graft or other permanent or semi-permanent medical device of claim 31, wherein the biodurable polymer is PVDF-HFP.

38. The stent, stent graft or other permanent or semi-permanent medical device of claim 31, wherein a weight ratio of the biodurable polymer to the therapeutic agent is from 1:1 to 10:1.

39. The stent, stent graft or other permanent or semi-permanent medical device of claim 31, wherein the biodegradable polymer is PLGA.

40. The stent, stent graft or other permanent or semi-permanent medical device of claim 31, wherein the excipient is sodium docusate.

41. The stent, stent graft or other permanent or semi-permanent medical device of claim 31, further comprising an antioxidant.

42. The stent, stent graft or other permanent or semi-permanent medical device of claim 41, wherein the antioxidant is chosen from probucol, vitamin E, vitamin E succinate, butylated hydroxytoluene (BHT), ascorbic acid, beta carotene, lycopene, lutein, retinol, manganese, selenium, flavonoids, flavones, catechins, polyphenols, tannic acid, and/or zeaxanthin.

43-121. (canceled)

122. A stent, stent graft or other permanent or semi-permanent medical device for delivering a therapeutic agent to a blood vessel, comprising a device body and a drug coating thereon, wherein the drug coating comprises:
a therapeutic agent and at least one of a biodurable polymer, and an excipient, wherein:
the therapeutic agent comprises a PDE inhibitor, a kinase inhibitor, an anti-fibrotic drug, or mixtures thereof,
the biodurable polymer is chosen from poly(vinylidene hexafluoropropylene) (PVDF-HFP), polyethylene terephthalate (PET), nylon 6,6, polyurethane (PU), polytetrafluoroethylene (PTFE), polyethylene, polysiloxanes (silicones) and poly(methylmethacrylate) (PMMA), and combinations thereof; and
an excipient chosen from a fatty acid, a fatty acid ester, polylactic acid (PLLA,PDLA,PDLLA), polycaprolactone (PCL), sodium docusate, PLGA, PLGA-b-mPEG, polyglutamic acid, polyacrilic acid, hyaluronic acid, alginate, PVA, PVP, Pluronic (PEO—PPO-PEO), cellulose, CMC, HPC, starch, chitosan, human serum albumin (HSA), phospholipids, fatty acid, fatty acid esters, triglycerides, beeswax, cyclodextrin, Tween 20, Tween 80, TPGS, SLS, butylated hydroxytoluene, vitamin E, vitamin E succinate, and sorbitol esters.

123. The stent, stent graft or other permanent or semi-permanent medical device of claim 122, wherein the PDE inhibitor is chosen from xanthine, aminophylline, sildenafil, tadalafil, vardenafil, udenafil, avanafil, dipyridamole, quinazoline, paraxanthine, papaverine, mesembrenone, rolipram, ibudilast, piclamilast, luteolin, drotaverine, roflumilast, apremilast, crisaborole, inamrinone, milrinone, enoximone, anagrelide, cilostazol, pimobendan, erythro-9-(2-hydroxy-3-nonyl) adenine), (2-[(3,4-dimethoxyphenyl)methyl]-7-[(1R)-1-hydroxyethyl]-4-phenylbutyl]-5-methyl-imidazo[5,1-f][1,2,4]triazin-4 (1H)-one), oxindole, (9-(6-phenyl-2-oxohex-3-yl)-2-(3,4-dimethoxybenzyl)-purin-6-one), 3-isobutyl-1-methylxanthine, pentoxifylline, theobromine, and theophylline.

124. The stent, stent graft or other permanent or semi-permanent medical device of claim 122, wherein the PDE inhibitor is tadalafil or sildenafil.

125. The stent, stent graft or other permanent or semi-permanent medical device of claim 122, wherein the PDE inhibitor is in the form of a free base, a crystal, a free acid or a salt.

126. The stent, stent graft or other permanent or semi-permanent medical device of claim 125, wherein the salt is of a hydrochloride salt, a sodium salt, a sulfate salt, an acetate salt, a phosphate and/or diphosphate sale, a pamoate or hemi-pamoate salt, a chloride salt, a potassium salt, a maleate salt, a calcium salt, a citrate salt, a mesylate salt, a nitrate salt, a tartrate salt, an aluminum salt, a stearic acid salt, a dioctyl sulfosuccinic acid or a gluconate salt.

127. The stent, stent graft or other permanent or semi-permanent medical device of claim 122, wherein the kinase inhibitor is chosen from bosutinib, ceritinib, crizotinib, gefitinib, ruxolitinib, imatinib, axitinib, nilotinib, trametinib, afatinib, ibrutinib, cabozantinib, imatinib, lenvatinib, sunitinib, regorafenib, sorafenib, vandetanib, dasatinib, pazopanib, Y27632, CA3, verteporfin, VGLL4 peptide, nintedanib, avapritinib, abemaciclib, erdafitinib, fedratinib, palbociclib, and pemigatinib.

128. The stent, stent graft or other permanent or semi-permanent medical device of claim 122, wherein the kinase inhibitor is sunitinib.

129. The stent, stent graft or other permanent or semi-permanent medical device of claim 122, wherein the kinase inhibitor is in the form of a free base, a crystal, a free acid or a salt.

130. The stent, stent graft or other permanent or semi-permanent medical device of claim 129, wherein the salt is of a hydrochloride salt, a sodium salt, a sulfate salt, an acetate salt, a phosphate and/or diphosphate sale, a pamoate or hemi-pamoate salt, a chloride salt, a potassium salt, a maleate salt, a calcium salt, a citrate salt, a mesylate salt, a nitrate salt, a tartrate salt, an aluminum salt, a stearic acid salt, a dioctyl sulfosuccinic acid or a gluconate salt.

131. The stent, stent graft or other permanent or semi-permanent medical device of claim 122, wherein the anti-fibrotic drug is chosen from triamciclone, tranilast, halofuginone, montelukast, zafirlukast, pirfenidone, nintedanib, and combinations thereof.

132. The stent, stent graft or other permanent or semi-permanent medical device of claim 122, wherein the biodurable polymer is PVDF-HFP.

133. The stent, stent graft or other permanent or semi-permanent medical device of claim 122, wherein a weight ratio of the biodurable polymer to the therapeutic agent is from 1:1 to 10:1.

134. The stent, stent graft or other permanent or semi-permanent medical device of claim 122, wherein the biodegradable polymer is PLGA.

135. The stent, stent graft or other permanent or semi-permanent medical device of claim 122, wherein the excipient is sodium docusate.

136. The stent, stent graft or other permanent or semi-permanent medical device of claim 122, further comprising an antioxidant.

137. The stent, stent graft or other permanent or semi-permanent medical device of claim 136, wherein the antioxidant is chosen from probucol, vitamin E, vitamin E succinate, butylated hydroxytoluene (BHT), ascorbic acid, beta carotene, lycopene, lutein, retinol, manganese, selenium, flavonoids, flavones, catechins, polyphenols, tannic acid, and/or zeaxanthin.

138. (canceled)
